SEQUENCE LISTING (43 sequences)

SEQ ID NO: 1 - DNA, 20 bp, Artificial Sequence
  modified_base (12)..(18): i
aayaaytgggcnaarggnca

SEQ ID NO: 2 - DNA, 20 bp, Artificial Sequence
  modified_base (15): n = a, c, g and/or , t/u
tccatytcrtccatnccytc

SEQ ID NO: 3 - DNA, 874 bp, Sclerotinia sclerotiorum
aaatcacccactctctcggtggtggaactggtgccggtatgggtacgcttttgatttccaagatccgtgaggagttcccagatcgtatgatggctaccttctccgtcgtcccatcgccaaaggtttccgataccgtcgtcgagccatataacgctactctctctgttcatcaattggtcgagaactctgacgagaccttctgtatcgacaacgaggctctctacgacatttgcatgagaaccttgaagctcagccacccatcctacggagatcttaaccacttggtctccgctgtcatgtccggtgttaccacctgtctccgtttccctggtcaacttaactcagatctccgaaagttggctgtcaacatggttccattcccccgtcttcatttcttcatggttggatttgctcctttgaccagtcgtggcgcacactctttccgtgctgttactgttccagagttgacccaacaaatgtatgatcctaagaacatgatggccgcttccgatttccgtaacggtcgttacttaacctgctctgctatcttgtaagtttgccatattacccgtctgcagctctatatatactaatcgtgtgcagccgtggtaaggtttccatgaaggaggttgaggaccaaatgcgcaatgtccaaaacaagaactcttcctacttcgtcgagtggatccctaacaatgtccaaaccgccctttgctccattcctccccgtggtctcaagatgtcctccaccttcgtcggtaactcgacctccatccaagaactcttcaagcgtgtcggtgatcaattcactgctatgttcagaagaaaggctttcttgcattggtacactggtgaaggtatggacgagatggag

SEQ ID NO: 4 - DNA, 1344 bp, Erysiphe pisi
atgcgcgagatcgttcacctccagactggccaatgcgggaaccaaattggagctgctttctggcaaaccatatctggagaacatggccttgacggatctggtgtatacaatggaacttccgacttacaacttgaaagaatgaatgtttatttcaatgaggcatccggaaataaatatgttcctcgagctgttctcgtcgacttagaacctggcactatggatgctgttcgagccggcccattcggtcagctctttagacccgacaattttgtattcggtcaatctggagctggtaacaactgggctaagggccattataccgagggtgcggagcttgtggatcaagtcttagatgttgtccgccgtgaagccgaagcttgtgattgtcttcagggttttcagataactcactcacttgggggtggaactggtgccggtatgggcacacttctgatatcgaaaattcgtgaagaattccctgatcgtatgatggctacattttcagttgttccatcaccaaaagtttcagatactgttgtggagccctacaacgcaaccctttcagttcatcaacttgtcgaaaactctgacgagactttctgtattgacaatgaagcactctatgaaatatgtatgagaactttaaagctctcgaatccatcttatggtgatcttaatcatttggtatctgccgtcatgtcaggtgtaaccacttgtcttcgtttcccaggtcaactcaactcagatcttagaaaattggcggttaacatggttccatttcctcgtcttcacttttttatggttggttttgctccacttaccagtcgcggtgcccactcattccgggctgtcactgtccccgagttaactcagcaaatgtacgatcccaaaaatatgatggctgcttcagacttccgaaacgggcgttatttgacatgctccgctatattccgtggtaaggtatcaatgaaggaagtggaggatcaaatgcgtaacgtacaaaataaaaactcagcatattttgtcgagtggattcccaataatgtgcaaactgcactttgctctattccacctcgtggtcttaagatgtcatctacatttgtaggaaactctacttccatccaagaactatttaagcgcgttggggatcaatttacagctatgttcaggagaaaagctttcttacattggtacactggagaaggaatggatgaaatggagtttactgaagctgagtcgaacatgaatgatttagtccacgaatatcaacaataccaagatgcctcgatttccgaaggtgaggaggaatacgaagaagagcagcaacttgaaaatgaggaatga

SEQ ID NO: 5 - DNA, 2475 bp, Blumeria graminis
  modified_base (37)..(137): n = a, c, g and/or t/u
cccggggcaaatcacactctgcctctctagcctcctncccgaaggtcgtgctgaaatttctggaaacagcgtaattgctgtatggtagcttagcccaactttttttgtgccgtccagggctctagggagtgctgttnagctagtgagaatagaagtcttccgagatatttgaaagcctaccatagctctgaaggcattgtggcaggacctagaggatcgtaagagatatgactgacaagtgttgagtccttgcgtcctaattttgtgtattattaccgttgtgagacatcacggcgacgcagaccgatctgcacttttcagtgccaagatggtatgtaagcagtcccgtgatcggagctagcgcagcaacggtttagtgtgagccaggtccaccgtcaacccgcaattactttctcgctgcgccaaatctccaatttctacatcccaactaacctccggaaacgcacctacactataccccctatcatcctctaccgcctatctttcatcagccaatatgcgtgaaattgttagttaaattccatcacggcaactcttgaaatcgctaatgataatttaggtccatttacagaccggtcaatgcgtacgttaatatttagtttgccttgtaactctactaatagagttttagggaaaccaaatcggagccgcattctgttcgtagaatctcaagcttctagctcaactcgctcacacccactctacagggcaaacaatttctggtgagcatggacttgatggttcaggagtgtaagttccctcgcataattaggtatccgcattcatcaacgaattataatgactccattagctacaatggtacatcagatttacaacttgagaggatgaacgtatactttaacgaggtctggtgaagctctcaaaagagaagttatcgctaaccctcttaaggcttctggtaacaaatatgttcctcgtgctgttctcgtcgacttggagccaggtactatggatgctgtacgagctggtccttttggccagctattcagaccagacaacttcgtcttcggacaatctggagccggaaataattgggcaaaaggtcactacactgaaggcgcggagcttgtcgaccaagtactagatgtagtgcgaagagaagcagagggatgtgactgtcttcaggggtttcaaataacacattctcttgggggtggtacaggtgccggtatgggtacgttattaatttcaaaaatccgggaagaattccctgatcgaatgatggcaaccttctcagttgtgccgtcccctaaggtgtctgacactgttgttgagccatacaacgcaactctttcagtccatcagttggtcgaaaactccgacgagactttctgtatcgacaatgaggcactttacgacatctgtatgaggacgttaaagctttctaacccctcatatggtgatctaaatcacttggtatccgctgtaatgtcaggcgtaactacttgccttcgattccctggccagctaaactctgatttgcgcaaactggcagttaacatggttcctttcccacgtcttcacttttttatggttggatttgcaccgcttacaagccgtggagcgcactctttccgtgccgtaactgttcctgagttaactcagcagatgtttgatccgaaaaatatgatggcagcatccgacttccgaaatggtcgttacttgacatgctctgccattttgtaagtcagctcgttatatacgcatattctatactaacatattacagccgcggtaaggtatctatgaaggaagtagaagatcagatgcgaaatgtccaacaaaagaacgtatcctactttgttgagtggattccaaataatgttcaaactgccctatgttctatcccgcctcgtggcctaaaaatgtcttcgacattcgtcggaaactcgacttccatccaagagctcttcaaacgtgtcggagatcaattcacggccatgttccggagaaaggcatttctgcattggtacactggcgaaggaatggacgaaatggagtttactgaggccgagtctaatatgaacgatcttgtctcagagtatcaacaataccaagaagcttcaatctcggagggtgaggaagagtatccagaggaagtaagcaacgaagaagaatagatcaaaattgtccttttaacacattgttctgtactgtatctgaagtagcggatacgatgaactgtaccccattaatcgtaccgttcctcttgtttggaaaattaaaagtcaccaaccaaaatgcgatagaaagtgccggtgtactggcctagcacatccttgtctgggtggatgtcgaacactttatttgctccattagctagtctctttccgcgcgacaggatgaaaactttctaatcatcaatcacatttcttttctacgaaagtttcaattctgttactgtagagttttttttgtaaaaccatctctaga

SEQ ID NO: 6 - DNA, 2632 bp, Gibberella fujikuroi
aactgagcttcctagcacctatataagctagattctataggtagattcttctttatctctgctgtagtataagaatttaccacatacatacatacctaccttggttgtttatatgcatctggagataaactggcgggctgctaccttatttcctcctctccatcggcagtgacaggtacggattcattggcgtgcctgccactgagggaagcccttcagcggcgccttgcccttcttggatcgcgtgcacggaccaagaggtaaacaactgagagtgcttactggtccaatagcctggaaggaaccgccctgccccttgtttaccccctgctgggccctgaagccgtggcggtgaacaatttcaccctcttgactgaggtgccctccagctccaaacaaaaatcaacatcggccctttggtctctttccaacccaacccaacccaacccactcttccctccgtcaaagctaggtctacttcagctcgacgacttcggcccatttctctcctccttctctctactcctccagattacattcatccacatccatcaacatgcgtgagattgtaagtacctctcttttttaagttcgtgctgtgctgttgcacgcgttgcgtttgtcgtgcccctgattctaccccgctgggcggtggcagctcaacgacaatgcatgatagatagctagcagctttcacataccttctgtcaagacgaagaagctaatcagatcttttctctacgataggttcacctccagaccggtcagtgcgtaagtgctcatcgcttcctcagcgtcgcatgaggggggatacttacagtgtttatcagggtaaccaaattggtgctgctttctggcaaaccatctctggcgagcacggcctcgacagcaatggtgtctacaatggtacctccgagcttcagctcgagcgcatgagtgttaacttcaacgaggtatgcattagcagtcaatgtcaagagttcacacgctcacacatctagcgctctggcaacaagtatgttccccgagccgtcctcgtcgacctcgagcctggtaccatggacgccgtccgtgctggtcccttcggtcagctcttccgtcccgacaacttcgttttcggtcagtccggtgctggaaacaactgggccaagggtcactacactgagggtgccgaacttgtcgaccaggttctcgacgtcgtccgccgtgaggctgagggctgcgattgcctccagggtttccagatcacccactctctcggtggtggtaccggtgccggtatgggtactctgctcatttccaagatccgcgaggaattccctgaccgaatgatggccaccttctccgtcgttccctcccccaaggtctctgacaccgtcgttgagccctacaatgccaccctctccgtccaccagctggtcgagaactccgatgagaccttctgtatcgataacgaggccctctacgatatctgcatgcgcaccctgaagctgtccaacccctcctacggtgacctcaactacctcgtttctgctgttatgtccggtgtcaccacctgtctccgtttccccggtcagctgaactccgatctccgaaagctcgccgtcaacatggtgcctttccctcgtctacacttcttcatggttggatttgctcctctgaccagccgtggtgctcactctttccgcgctgtcagcgttcctgagttgacccaacagatgttcgaccccaagaacatgatggctgcttcggacttccgcaatggtcgctacctgacctgctcagccattttgtgagtgaacccgatttgcgcatggaaattatttactgactttgaacagccgtggccgtgtcgctatgaaggaggtcgaggatcagatgcgcaacgtccagaacaagaactcttcttacttcgttgaatggattcccaacaacatccagacagccctttgtgccatccctccccgaggacttacgatgtcttcgaccttcatcggaaactccacttctatccaggagctcttcaagcgtgttggtgagcagttcactgccatgttccgacgcaaggctttcttgcattggtatactggtgagggtatggacgagatggagttcactgaggctgagtccaacatgaatgatcttgtctctgaataccagcagtaccaggatgctggtattgatgaggaggaagaggagtacgaggaggagctccctgagggcgaggagtaaatctactccgcagtgtgctgaaataactggccgctatgttatgtttgtcgtatagccggcccgcaacttcttttcgagtgtagttgttgtaattctggggtaaagatatcctcaatagtggcagtacaccatagttgtatgagccgaataaaattccaagagtactattgtaccaattcaattctcaaagatggtgaaactagcactgacttactcgaactggtcacaagccagtttcgccgaggactcgaaatgtttctgtgacatgaagttaagcatcggcaatcggaccacattgaccgacaggcccttggtgtgcagtaggtagtcatcatgctgagcacgtgatgtcatgactaagcgcgaaaagttgagctccatcagcttgtcaacctcattc

SEQ ID NO: 7 - DNA, 1344 bp, Gibberella zeae
atgcgtgagattgttcaccttcagaccggtcagtgcggtaaccaaatcggtgctgctttctggcagaccatctctggcgagcacggtctcgacagcaatggtgtttacaacggcacctctgagctccagctcgagcgcatgaacgtctacttcaacgaggcctcccgtaacaagtatgttccccgtgccgtcctcgtcgatctcgagcccggaaccatggacgccgtccgtgctggtcccttcggacagcttttccgacccgacaacttcgttttcggtcaatccggcgccggaaacaactgggccaagggtcattacaccgagggtgctgaacttgtcgaccaagttctcgatgtcgtccgccgtgaggccgagggctgtgactgcctccagggtttccaaatcacccactctcttggtggtggtaccggcgccggtatgggtaccctgttgatctccaagatccgtgaggaattccccgaccgtatgatggcaactttctccgtcgttccttcccccaaggtctccgacaccgttgtcgagccctacaacgccaccctctccgtccatcaattggtcgagaactccgacgaaactttttgtatcgataatgaggccctctacgacatttgcatgcgcaccctcaagctgtccaacccctcttacggcgacctgaactaccttgtctctgccgtcatgtccggcgtcactacctgtctccgtttccccggtcagctgaactctgacctccgaaagctcgccgtcaacatggtgcccttcccccgtctgcacttcttcatggtcggattcgctcccttgaccagccgtggtgctcactctttccgcgctgtcagcgttcctgagctcacccagcagatgttcgaccccaagaacatgatggctgcctccgacttccgcaacggtcgttacctgacctgctctgccatcttccgtggccgtgtcgccatgaaggaggttgaggaccagatgcgcaacgtccagagcaagaactcatcatacttcgtcgagtggattcctaacaacatccagaccgctctctgcgctattccccctcgtggacttactatgtcttccacttttattggaaactccacctctatccaggagcttttcaagcgtgttggcgagcagtttactgctatgttccgacgcaaggctttcttgcattggtacactggtgagggtatggatgagatggagttcactgaggccgagtccaacatgaacgatcttgtctctgaataccagcagtaccaggatgctggaattgacgaggaagaggaagagtacgacgaggaggagctccttgagggcgaggagtaa

SEQ ID NO: 8 - DNA, 1344 bp, Aspergillus nidulans
atgcgtgagatcgttcaccttcagaccggccagtgtggtaaccaaattggtgctgctttctggcagaccatctccggtgagcacggcctcgatggctccggtgtttacaatggtacctccgaccttcaactcgagcgtatgaacgtctacttcaatgaggccagcggtaacaagtacgttccccgtgccgtcctcgtcgatctcgagcccggtactatggatgccgtccgcgccggtcccttcggcgagctcttccgtcccgacaacttcgttttcggccagtccggtgctggtaacaactgggccaagggtcactacactgagggtgctgagcttgttgacaacgtcgtcgatgttgtccgtcgtgaggccgagggttgcgactgcctccagggtttccagatcacccactctctcggtggtggtaccggtgccggtatgggtactcttttgatctccaagattcgtgaggagttccccgaccgcatgatggccaccttctccgtcgttccctctcccaaggtctccgacaccgttgttgagccttacaacgccaccctttccgttcaccagctcgttgagcactccgatgagactttctgtattgacaacgaggctctctacgatatctgcatgcgcaccctcaagctctccaacccctcctacggtgatctgaaccacctcgtctccgccgtcatgtccggtgtcaccacttgccttcgattccctggtcagctgaactctgacctgcgcaagctggctgtcaacatggttcccttccctcgtctgcacttcttcatggtcggcttcgctcctctgaccagccgtggcgcctactccttccgcgctgtttccgttcccgagttgacccagcagatgttcgaccccaagaacatgatggctgcctctgacttccgcaacggccgctacctcacctgctccgctatcttccgtggaaaggtctccatgaaggaggttgaggaccagatgcgcaacatccagagcaagaaccagtcctacttcgtcgagtggattcccaacaacatccagaccgctctctgctccattcctccccgcggcctcaagatgtcttccaccttcattggaaactctacttccatccaggagctcttcaagcgtgtcggtgaccagttcactgctatgttccgtcgcaaggctttcttgcattggtacactggtgagggtatggacgagatggagttcactgaggctgagagcaacatgaacgatctcgtctccgagtaccagcagtaccaggacgcctccatctccgaaggcgaggaggagtacgccgaggaggagatcatggagggtgaggaataa

SEQ ID NO: 9 - DNA, 1344 bp, Neurospora crassa
atgcgtgaaattgttcatctccaaaccggccaatgcggtaaccaaatcggtgctgctttctggtacactatctccggcgagcacggcctcgatgcctccggtgtgtacaatggcacctctgagctccagctcgagcgcatgaacgtctacttcaacgaggcttccggcaacaagtatgtccctcgtgccgtcctcgtcgatctcgagcccggtaccatggacgccgttcgcgccggtcccttcggccagctcttccgccccgataacttcgtcttcggccagtccggtgctggcaacaactgggccaagggtcactacactgagggtgctgagcttgttgaccaggttctcgatgtcgtccgtcgcgaggctgagggctgcgactgcctccagggcttccagatcacccactccctcggtggtggtaccggtgccggtatgggtaccctccttatctccaagattcgtgaggagttccccgaccgcatgatggctaccttctccgtcgtgccctcccccaaggtctccgataccgttgtcgagccctacaacgccaccctctccgtccatcagctcgttgagaactccgacgagaccttctgcattgacaacgaggcgctttacgacatttgcatgaggaccctcaagctctccaacccctcttacggcgatcttaaccacctcgtctccgccgtcatgtccggtgtcaccgtctccctccgtttccccggccagctgaactccgatctccgcaagctcgccgtcaacatggttcccttcccccgtctccacttcttcatggtcggcttcgctccccttaccagccgcggcgcccaccacttccgtgccgtctccgtgcccgagttgacccagcagatgttcgaccccaagaacatgatggctgcttctgacttccgcaacggtcgttacctcacctgctctgccatcttccgtggcaaggtctccatgaaggaggttgaggaccagatgcgcaacgttcagaacaagaactcttcctacttcgtcgagtggatccccaacaacgtccagactgccctctgctctatccctccccgcggtctcaagatgtcctccaccttcgtcggtaactccaccgccatccaggagctcttcaagcgtatcggcgagcagttcactgccatgttcaggcgcaaggctttcttgcattggtacactggtgagggtatggacgagatggagttcactgaggctgagtccaacatgaacgatctcgtctccgagtaccagcagtaccaggatgctggtgttgacgaggaggaggaggagtacgaggaggaggccccccttgagggcgaggagtaa

SEQ ID NO: 10 - DNA, 2182 bp, Aspergillus flavus
ccccttgacgcccgcacaacgaacaacttgacgttcctcaccgctcaacttcaaggccatctttcctccttctctcttctcctcttccttttacctactccccgtcgactgtctcccccagtctatccaacaacccttctccaacgacctcttcgccgttttcaaacccaccttttcctaccaacaacgccaaaatcccctccacaatgcgtgagatcgtatgttgctccctacccccggtggggggagaagtctgctcaaaaagccctatcccccccccctgatagggaccccacccgttctccaatactacaaggttgctgacggagtttgtttcatcatataggttcaccttcagaccggccagtgtgtaagttcgactatgatttgatgtctagcaggaccatggcgacggatactaaacgtatgttggtgatagggtaaccaaataggtgccgctttctggtatgtctcaatgccttcgagttagtatgctttggaccaaggaactcctcaaaagcatgatctcggatgtgtcctgttatatctgccacatgtttgctaacaactttgcaggcaaaccatctctggcgagcacggccttgacggctccggtgtgtaagtacagcctgtatacacctcgaacgaacgacgaccatatggcattagaagttggaatggatctgacggcaaggatagttacaatggctcctccgatctccagctggagcgtatgaacgtctacttcaacgaggtgcgtacctcaaaatttcagcatctatgaaaacgctttgcaactcctgaccgcttctccaggccagcggaaacaagtatgtccctcgtgccgtcctcgttgatcttgagcctggtaccatggacgccgtccgtgccggtcccttcggtcagctcttccgtcccgacaacttcgttttcggccagtccggtgctggtaacaactgggccaagggtcactacaccgagggtgccgaacttgttgaccaggttgtcgatgttgtccgtcgcgaggctgagggctgcgactgcctccagggtttccagattacccactccctcggtggtggtaccggtgccggtatgggtactctcctgatctccaagatccgtgaggagttccccgaccgtatgatggccacctactccgttgtcccctcccccaaggtctccgacaccgttgttgagccctacaacgccactctttccgtccaccagcttgttgagcactccgacgagaccttctgtatcgacaacgaggctctgtatgacatttgcatgcgcaccctcaagctctccaacccctcttacggtgacctgaaccacctggtctctgctgtcatgtctggcgtgaccacctgtctccgtttccccggtcagctcaactctgatcttcgcaagttggccgtcaacatggttcctttccctcgtcttcacttcttcatggttggcttcgctcctctgaccagccgcggtgcccactctttccgtgccgtctccgttcctgagttgacccagcagatgttcgaccccaagaacatgatggctgcttctgacttccgtaacggtcgttacctcacctgctctgctatcttgtgatgtggcccctattttctatttgttctatcctctgttgtttgaaaactgacctttcgatagccgcggaaaggtctccatgaaggaggttgaggaccagatgcgcaacatccagagcaagaaccagacctacttcgtcgagtggatccccaacaacatccagaccgccctgtgctccattcctccccgtggtctcaagatgtcctccaccttcattggaaactccacctccatccaggagctcttcaagcgtgtcggcgaccagttcactgctatgttccgtcgcaaggctttcttgcattggtacactggtgagggtatggacgagatggagttcactgaggctgagagcaacatgaacgaccttgtctccgagtaccagcagtaccaggatgcctccatctccgagggcgaggaggaatagtaaggattcccattggccctgctctcgtgtatttgtgctaaccagtttgcagcctcgaggaggaggagccccttgagcacgaggagtaaatagcttccagtcactaaagactcggattgatatctggcagcaatacccttgataagtcca

SEQ ID NO: 11 - DNA, 1344 bp, Magnaporthe grisea
atgcgtgaaattgttcaccttcagaccggccaatgcggcaaccaaattggtgctgctttctggcaaactatctctagcgagcacgggctcgacagcaatggagtttacaacggcacctccgagctccagctggagcgtatgagcgtctacttcaacgaggcctccggcaacaagcatgttccccgtgctgtcctcgtcgatctcgagcccggcaccatggacgccgtccgtgccggtccttttggccagctcttccgccccgacaacttcgtcttcggtcagtctggtgctggaaacaactgggccaagggtcactacactgagggtgccgagcttgtcgaccaggtccttgacgtcgtccgtcgtgaggctgagggctgtgactgcctccagggtttccagatcacccactccctgggtggtggtaccggtgccggtatgggtactctgctgatctccaagatccgcgaggagttccccgaccgtatgatggccaccttctcggtcgttccctcgcccaaggtttccgacaccgtcgttgagccctacaacgctaccctctcggtccaccagctggtcgagaactctgacgagaccttctgcattgacaacgaggctctgtacgacatctgcatgcgcaccctgaagctgtcgaacccctcatacggtgacctgaactacctggtttcggccgtcatgtctggcgtcaccacctgcttgcgtttccccggccagctcaactctgatctccgcaagcttgccgtcaacatggttcccttccctcgtctgcacttcttcatggttggcttcgctcctttgaccagccgtggtgcccactctttccgcgctgtcaccgttcccgagttgacccagcagatgttcgaccccaagaacatgatggctgcttctgacttcaggaatggtcgttacctgacctgctctgccatcttccgtggaaaggtttccatgaaggaggtcgaggaccagatgcgcaacgtccagaacaagaactcgtcgtacttcgtcgagtggatccccaacaacatccagaccgctctctgctctatcccgccccgcggcctcaagatgtcgtcgactttcatcggaaactcgaccgccatccaggagctgttcaagcgtgtcggtgagcagttcactgccatgttcaggcgcaaggctttcttgcattggtacactggtgagggtatggacgagatggagttcactgaggccgagtccaacatgaacgatcttgtttccgagtaccagcagtaccaggatgctggtgttgacgaggaggaagaggagtacgaggaggaggcccctcttgagggcgaggagtag

SEQ ID NO: 12 - DNA, 3229 bp, Epichloe typhina
ctgcagccgcctgcccagccgggggccctgccaagtctggcggtgaaccgaccggctgtccaattcatctccaacgaaaaatcaacaacgtccgccccggcttagtcctctcttgttcccatacattacaccttctggcggaccgtgaagcttcgcctggctctcgtgattcttttcatcattattcttttcccagcttcagaagacttgccgtcgtcaacaagtaaccgagaaaatgcgtgagattgtgagttcaacctctctgtttgtcttggggaccccctcctcgacgcgttcccgtgttgagcccctgatttcgtaccccgccgagcccggccacgacgtgcacgcccaacggacaagtcgtgatgagaggcggaccgagacaacattgatgaatgcggtattcgaaaaccgtagctgaccttttttctttgcctctaggttcatcttcaaaccggtcagtgcgtaagtgacaaatccgccgacctcgaacgacaggcacaaataacatgaaaaactcacattgatttgggcagggtaaccaaattggtgctgctttctggcagaccatctctggcgagcacggcctcgacagcaatggtgtgtacaatggtacctccgagctccagctcgagcgtatgagtgtctacttcaacgaggcaagtcttcataatctacaaaagtctccattgagctacatactgccctggagatggggcgggagaagatggaaaggaaaagtgttatcatgctaatctatgtgacaggcttctggcaacaagtacgttcctcgcgctgttctcgtcgatctcgagcctggtaccatggatgcagtccgtgccggtcccttcggtcagcttttccgtcctgacaacttcgtcttcggtcagtctggcgctggcaacaactgggccaagggtcactacactgagggtgctgagctggttgaccaggtcctcgatgttgtgcgtcgcgaggccgaaggctgtgactgcctccagggtttccagatcacccactctcttggtggtggtaccggtgccggtatgggtacattgttgatctccaagatccgtgaggagttccccgaccggatgatggctactttttccgtcgttccctctcccaaggtctctgacaccgttgtcgagccctacaacgccactctctctgtccatcagcttgtcgagaactcggacgaaacgttctgtatcgataatgaggccttgtacgatatctgcatgcgtactcttaagctgtccaacccctcgtacggcgatttgaactacctggtctccgctgtcatgtctggcgtcaccacctgcctgcgtttccctggtcagctgaactctgacctgcgcaagttggccgtcaacatggttcctttcccccgtctgcacttcttcatggtcggcttcgcccccctgaccagccgtggcgcccactctttccgcgctgtcagcgtccctgagcttacccagcaaatgttcgaccccaagaacatgatggctgcttctgatttcagaaatggtcgctacctgacctgctctgccattttgtgagtgaaattgacaacctcaacttgagaagtcgtgtcgcagtaactgactgggaacaaacagccgtggcaaggtcgctatgaaggaggtcgaggaccagatgcgtaacgtgcagaacaagaactcttcctacttcgtcgagtggatccccaacaacatccagactgctctctgcgccatccctccccgtggcctcaagatgtcttctacctttatcggtaactccacctccatccaggagctcttcaagcgggttggcgagcagttcacagccatgttccgtcgcaaggctttcttgcattggtacactggcgagggtatggacgagatggagttcactgaggctgagtctaacatgaacgatcttgtttccgaataccagcagtaccaggatgctggtattgatgaggaagaagaggagtatgaggaggaggcacctgttgacgagcctctggagtaaggtgcctgttgggttatttgaaccccgccattgtatctcgttcaattaagcgtggcgtgagcaggtaaaatttacttcggtattgtaataactacattccaccccccctaaggaggggttaaaaaagatggcttttatcactgagtgactgtgctacattgggaagattgtacgtcatgagtcaaaaacaaagtaagttcttgttcttatttttgttttgaattacaaaatttgtgacgccaaggatacctgttgtatgtccgcttcttgtttttttttttttgaatttccaggaaatgactcaaatgagtgacgtgattaaggggatacgtacagcacgtgataagatgactaagcgtacgaaaagtatgggttgtcaatccgcgctgaccttcaatcacagtcagtacaacgaaacacgtcccatccattgcgacagcaaggacaggactcaatggcctcaaaaaccatgcctcgccggtgcaaggtgctgggacatgccttgggctcgtcttcgggctcagcctgccagggttgctcgtgccagcgtggccaacgtttgcgcaaagacaattctcgacgacgacaaagcgaccttcgaaacttggtcgaacgccgattaccatcccccccggcgtcgaattgtcgatgggcgacttgaagaaattcaagtccaagacgtcgtacaaacctaccgtgaatagaaggatcacggtcaaaggaccgttgggtgagttttcattggctcgaggtcgcagtggtggtggttctgcgctgccaccggaatgcaggtcaacttgtacggcaggacggacgtagctgaccaaagatgttttttttttttttttttcacagggaaactgggacttgatgttcctgaattcgtcagcctgcgcaggacttggagaacaagacggtgtcgttgggctcgaggatgcgaatgtcaagcagcagaaggagatgtggggtatgcattggacacaactttcccatggttcaacctttaaccctagaagtacaggtttgggggaaagcggtattgggttggagttcatactgactttgggtccgggcaggaacatcgtggtcgtacctgaccaaccacgtgaggggggtgtcggaaggccacacggccatactccgcctcgtgggcgtgggataccgagcgagcgtcgamgaccggggmmmmaaggagcagtaccccggg

SEQ ID NO: 13 - DNA, 2614 bp, Colletotrichum graminicola
cccgggtcttggatcggagagaacagaggagcagaatagggattattgggtaggcaggtacgtgcccaggtaacacgtcgtgcctgccaatcaacgccgatctgagtcctcgacttgttgcgacccaccatcaaatagtgcctgggaatggtctttccccaatcaggatcgtgcacggatcccaatagtaaacaagcgagctgcacccttttcctctctggcctgtctctgggcacctggcctgtggcggtgagcgaaatcacgtttacccgcaaaacaaaaatcaacaacctttcccctacctaaccacaccttaatctcatccactttccaaccacgtccaccttggaagcttcgcgcagctctcaagcacctcccactcgtcctctcctttgctccatcgtcggcctagtcagctcaagagcttttctccattcacaatgcgtgagattgtaagtccttcccctcaatcattcgtaacaaataaacctgcgaccgacgcgtttggcgacgaatcgtcggccttgcccctgaacgtaccccgccgacatttccacccaacactggtcctcaccgaagacgaccacgattgccatcaccgacagtatgcaccttggggatatatcgctgaccattgattgttatactcgataggttcacctccagaccggccagtgcgtaagtcttctctgatcccaaccaacaatccaaggtgcggggctaacttctttgaatagggtaaccagattggtgctgccttttggtgcgtagccagaccgacatcatcgacttcggcgagattggcctcgaaaagacattggatattaatacgggcacaggcaaaacatctctggcgagcacggcctcgacagcaatggcgtgtatgttgccaacctccagatctggccacttcctcgagttcaccgctaatttctcaacagttacaatggcacctctgagctccagctcgagcgcatgagcgtctacttcaacgaagtttgttatcctagccccccccaggaagcagacaaacctattgatgaatactgaccttgtcacgtacccaggcttccggcaacaagtatgtccctcgcgccgtcctcgtcgacttggagcccggtaccatggatgctgttcgcgccggccccttcggccagcttttccgccccgacaacttcgtctttggccagtccggtgccggcaacaactgggccaagggtcactacaccgaaggagctgagcttgtcgaccaggtccttgacgtcgtccgccgcgaggctgagggctgcgactgccttcagggcttccagattacccactcccttggtggaggtactggtgccggtatgggtactctgttgatctccaagattcgcgaggagttccccgaccgcatgatggctaccttttccgtcgttccctcccctaaggtttccgacaccgttgttgagccctacaacgccactctctccgtccaccagctggttgagaactccgatgagaccttctgcattgataacgaggctctctacgacatctgcatgcgtactcttaagctctctaacccctcgtacggcgacctgaaccatctcgtctctgccgtcatgtccggtgtcactacctgcctgcgtttccctggtcagctgaactctgacctgcgtaagctggccgtcaacatggttcctttcccccgtcttcacttcttcatggtcggattcgctcccctgaccagccgtggtgcccactctttccgcgctgtcagcgttcctgagctcacccagcagatgttcgaccccaagaacatgatggctgcctctgacttccgcaacggtcgctacctgacctgctctgccatcttgtaagtgtcatttccagccaacctacaatgtcagcacttgctaacagctgcctctcagccgtggtaaggtcgccatgaaggacgtcgaggaccagatgcgcaacgtcctgaacaagaactcttcttacttcgtcgagtggatccccaacaatgtccagaccgccctctgctccattcctccccgcggcctcaagatgtccttcacctttgtcggtaactctaccgccatccaggagctcttcaagcgtgtcggtgagcagttcactgccatgttccgtcgcaaggctttcttgcattggtacactggtgagggtatggacgagatggagttcactgaggctgagtccaacatgaacgacttggtctctgagtaccagcagtaccaggacgctggtgttgatgaggaggaggaggagtacgaggatgacgcccccctggaggaggaggtttaagcgttgtctgaaaatgctgtgccaccttggccatgtcttcacccaacccgtctgcggtggcatttcgcttcactattccagctctgcactggaaatgggcttctagatatacctctcttagtagttcgcctggcgtatcaaaatgagtacgaagaatcagagattactctgtacaaattattggcaacatcaaatgcatagttttatggcaattgcgacacctctaatcttgccagagttcaagtattcgtatcttctttcgtgactgacagattatctatgtttaaacgtggacaccctcgag

SEQ ID NO: 14 - DNA, 3435 bp, Botryotinia fuckeliana
gatatccttgctctttgtcgattgacctgatcccaattgcaaaggattgacagccatgaagaactcttcatatgactgggcgtcatcgggttgattgataagtttagaagcctcatactcgacggccttgaattccttcttgctttgtttagcagcaatctttctttctgctttctccaacttctttttatcaaccctcgactctacttttctaacatttgcggattccaaatcaacacctccaagagatacacccaaagtagacgacatattcctctgagccccaacttggatggcttggtctagtcgtttggcagcggaagagagctgcttgttttgcccactgctatcaaatttttgaacccatttctccactatttgattgatctttgactgattctctggactcggatcaccagaagctgatagtaagacaggagatagcaacggctgcttctgataatggtgatggaccattttggtctgcctcgacatatgcatttgatgcatgggtgaggtacccaactgaatattccgatagaaccggatcaatgttgggtaagaaagctttgatttcgttctccatgatgattaatatgatacagaaagtaaaatatcaattcgtaccgccaacaaagggaccctctacacgaatcgactactttgatgcgaattgcctcacaatccgcaagtttattttcacgtgatctgcagttttccgctgtccgacttactaagcccgttgaggaaattaatccccactagcgttttatttgtttaccgattactcctgcctagcggtgaacaacaaactccactatccacaaggacatctcagcaatccttaacctcttaatacttctctatcctcaacctcgacttctcaatacccaaactcccatatctcaatacctcaacacaagatcctaaatcaaccttcaaaatgcgtgagattgtatgtatttctctctcttcatttacgatttctacgccttcttgcaagacgcgtcgactttacccctgaaaagcaccccactatatattttttaaaagtaacatatcgctgaccaagtaacttttcaatctacaggtccatcttcaaaccggccaatgtgtaagtaaacccatccaaatatattctatgagctttgctgacaatctgctcagggtaaccaaattggtgctgctttctggtacgagatctcggatctgcgaaacgtcttgcttcgcgacaacctcagattgcaactaaccatatcacaggcaaactatctctggcgagcacggtcttgacggttccggtgtgtaagtaaaatcacaaattcttctcgtacttgaaacgttactgatattgtttacagctataatggtacatccgatctccaacttgagcgtatgaacgtctacttcaacgaggtatatatacacaatttcgactctgctgaaaaccgtccgctaacccctataaggcttctggcaacaagtatgttccccgtgccgtcctcgtcgatttggagccaggtaccatggatgctgtccgtgccggtcctttcggtcaactcttccgtcccgacaacttcgttttcggtcaatctggtgctggtaacaactgggctaagggtcattacactgagggtgctgagcttgtcgaccaagttcttgatgttgtccgtcgtgaagctgaaggctgtgactgccttcaaggattccaaattacccactctctcggtggtggaactggtgccggtatgggtacgcttttgatctccaagatccgcgaggagttcccagatcgtatgatggctaccttctccgtcgtcccatcgccaaaggtttccgataccgttgtcgagccatataacgcaactctctctgtccatcaattggttgagaactctgacgagaccttctgtatcgataacgaggctctttacgatatttgcatgagaaccttgaagctcagcaacccatcttacggagatcttaaccacttggtttccgccgtcatgtccggtgttaccacctgtctccgtttccctggtcaacttaactcagatctccgaaagttggctgttaacatggttccattcccccgtctccatttcttcatggttggatttgctcctttgaccagtcgtggcgcacactctttccgtgctgtcaccgttccagagttgactcaacaaatgtacgaccctaagaacatgatggccgcttccgatttccgtaacggtcgttacttgacatgctctgccattttgtaagtttgccctgtaatcaatctgccaaaatcttgtagaaactaactttctgtagccgtggtaaggtttccatgaaggaggttgaggaccaaatgcgcaacgtccaaaacaagaactcatcctacttcgttgagtggatccctaacaacgtccaaaccgccctttgttccattcctccccgtggtctcaagatgtcctccaccttcgttggtaactcgacatccatccaagaacttttcaagcgtgtcggtgatcaattcactgctatgttcagaagaaaggctttcttgcattggtacactggtgaaggtatggacgagatggagttcactgaggctgagtccaacatgaacgatttggtttccgagtatcaacaataccaggatgcctcgatctctgagggagaggaggagtacgaagaggaggtcccaattgagggcgaggaatagatatcgttgagaatcgtttcatcggtctcaagtcccgtggatgttatgaaactcctggtctcacatgtctccgctccgcccacgttgatctcgaaggtttggttatggaccgtgaagtccgtgctcttcgtgaccaattcgtcacctacaactactctaagatcctttacaatggtctttacttctctcctgagcgtgagttcatcgaggaatctatcgttgcttcccagaagaatgtcaatggacaagtcagatgccgtgtgtacaagggtaccttcagtgtcttgggtcgttcctcagagaccgagaagttgtacgatgcaagcgagagttcaatggacgaaattggttcattcgctcctgcggatactactggtttcatcagcgttcaatctatcagattgaagaagtatggtgaggctaaggcagctgctggtgaaagactatagatggatcttgacaccataacggcttgaatttagtctgagtcttggattggacctgagacattcgggaacttgcaacatcgccacaaaatgcagatgagacaccgattgcctccggtcctcggagtgcagatgggaatgggaatatgtaaactaaaatgctacacaaaagtcgcatatgaatgaaaacagaccagcttgttattgccagtgccgcttcaattcatgatgatttatgtgtttgtttccaagaaaagacttaatgatgattactaacag

SEQ ID NO: 15 - DNA, 1666 bp, Leptosphaeria maculans
ggcacgagccaacaccaccctctaccaacttcttgcgcattctcagctcccacccaacacctttacctcagcaccacatcgcccaccgccatcatgcgtgagattgtccacctccagaccggtcaatgcggtaaccaaatcggtgccgccttttggcaaaccatctccggcgagcacggcctcgacggctccggtgtctacaatggcacttcagatctccagctcgagcgcatgaacgtctacttcaatgaggcttccggcaacaagttcgttccccgtgcggtcctcgtcgatctcgagcctggtaccatggatgccgtccgcgctggacctttcggacagctcttccgtcccgacaacttcgtcttcggccagtctggtgctggtaacaactgggcgaagggtcactacactgagggtgccgagctggttgaccaggtcctcgatgtcgtccgtcgcgaggctgagggctgtgactgcctccagggtttccagatcactcactccctcggtggtggtaccggtgctggtatgggtaccctattgatctccaagatccgtgaggagttccctgaccgtatgatggccaccttctcggtcgtgccctcccccaaggtctccgacaccgttgtcgagccctacaacgccaccctatccgtgcaccagcttgtcgagaactccgacgagaccttctgcattgacaacgaggctctctacgatatctgcatgaggaccctcaagctgaacaacccttcatacggtgacctgaaccacctcgtctccgccgtcatgtcgggtgtcaccacctgcctgcgtttccctggtcagctcaactccgatctcaggaagttggctgtcaacatggtgcccttcccccgtctccacttcttcatggtcggattcgctccccttaccagccgcggtgcccactccttccgcgccgtcaccgttcccgagctcacccagcagatgttcgaccccaagaacatgatggctgcgtccgacttccgtaacggtcgctacctgacctgctctgcctacttccgtggtaaggtctccatgaaggaggtcgaggaccagatgcgcaacgtccagaacaagaactcatcctacttcgttgagtggatccccaacaacgtccagaccgctctctgttccgtgcctccccgtggcctcaagatgtcctccacctttgtcggtaactcgacctccattcaggagctattcaagcgtgttggtgaccagttcactgccatgttcaggcgcaaggctttcttgcattggtacactggtgagggtatggacgagatggagttcaccgaggccgagtccaacatgaacgatctggtgtccgagtaccagcaataccaggaggcttccgtctccgacgctgaggaggagtacgacgaggaggctcctcttgaggctgaagagtaagcttgccaactgtaatacctcgctgggcttgagtattggtattggtggtctactataatgtttcgattcgtcctttgagcaagtcgtctgcatccaagtgattgtaatgaggcaagttcaacttgtttcggcacaatgggttgggtcaagcgacgtctcttgagtgtagaagcaaatcttgttagcgaaaagattatattgcatgcttctcaaaaaaaaaaaaaaaaaa

SEQ ID NO: 16 - DNA, 20 bp, Artificial Sequence
  Description of Artificial Sequence Synthetic Primer
athcargtntaygargarac

SEQ ID NO: 17 - DNA, 20 bp, Artificial Sequence
  Description of Artificial Sequence Synthetic Primer
ccytgrtcngcnggcatytc

SEQ ID NO: 18 - DNA, 1658 bp, Gibberella zeae
tccatttacattccccgcggtatcgccgttcctgccctcgaccgcgacaagaagtgggagttcacgccctccgtcaaggtcggcgaccacctctctggtggtgatgtttggggttccgtctttgagaactctttccttgccaaccataagattctgttccctccccgcgcccgaggaactgttaccaagatcgcctccaagggcgagtacactgtcgtcgacaacattctcgaggtcgagttcgatggcaagaagaccgagtaccccatgatgcagtcctggcccgtccgagtgccccgtccttccaacgacaagaagtcttccgatcagcctttcattgtcggccagcgagtcctcgatgcccttttccccagtgtccagggtgattcggccaaaaaccccccggtgctttcggtttgtggaaagactgtcattagtcagtctgtgtccaagttctcaaacagtgacgttattgtctacgtcggctgtggtgagcgaggtaacgagatggctgaagtcttgaaggatttccccgagctcaccattgaggttgatggccgcaaggagcccatcatgaagcggaccacacttattgccaacacttccaacatgcccgtcgcagctcgagaagcctccatttacacgggaattacagtggctgaatatttccgcgaccagggtctcaacgtcgccatgatggctgattcttcatcccgatgggctgaggctcttcgagaactttcaggtcgtctgggagaaatgcctgctgatcagggtttcccgcttacctgggtgccaagcttgcctcgttctacgagcgagccggtcgtgtccagacacttggttctcctgagcgcgagggtagtgtcagtatcgtcggtgctgtcagtccccctggtggtgatttctccgatcccgttacaacagccactctgggtattgtgcaggtcttctggggtcttgacaagaagcttgctcagcgaaagcatttcccttccattaacacctcggcctcttacagcaagtacaacaacattctggacaagtactacgagaagaactaccctgatttcccccgactccgcgaccgtatcaagcaactcctttccgactctgaggagctcgaccaggtcgtgcagcttgtcggaaagagtgctctgtctgatcccgacaagattactctcgatatcgctggcttgatcaaggaagatttcctccagcagaacggttactcagactatgaccagttctgccctatctggaagactgagtggatgatgaagctcatggtcggtttccacgatgaggctcagaaggttattgcacagggtcagagctgggccaaggtgcgagaggctacatccgaactacaagccaacctacgacagcttaagttcgaggtgcctaccgatggccaggaagttattaccaagagagtaagttaccagtcatctatgacatttgatatgcatacactaacacggcttttagtacgaggagatccaacagaagatgacagacaagttcgctgctgtcatggacgagtaagacctgagcaagctgctggttatttctgtcttgaagctatagatgagtcatgttgtggaataggccagaacatggctagtattcgattactctttgcccgccccgtatct

SEQ ID NO: 19 - DNA, 1818 bp, Aspergillus oryzae
atggccccctccggaaagggctctcaagacgaagcccaccatggcgccgtcttctccgtctccggtcccgtcgtcgtggccgagaacatgatcggctgtgctatgtacgaattgtgtcgtgtcggaaaagatcagctcgtcggtgaagttatccgtctcgatggcgataaggccactattcaggtctacgaggaaactgatggtgtgacggtcggcgaccctgtcgagaggacgggtaaacccctagcggtggaactgggtcctggttttatggaaactatctacgacggtatccaatgccccctgaaagctatctttcaccagtccaaaggaatctacatcccccgtggtatcactgtaaacgcgctggatcgcgagaagaaatgggacttcacgcccggtcaatacaaagtgggcgaccacatcactggtggtgatgtctggggttccgtgttcgagaacagcttgttgaacgaccataagatccttctcccgccccgcgctcggggtaccattacccgtatcgcagaggctggaagctacacagtcgaggagaaacttttggagatcgaatttaacggcaagaagtccgagtttggtatgatgcagacctgggccgtccgtgtacccgaccagtcaacgataggttccatcgacgcacccttcatcgtcggccagagagtgctggactctctgttccctagtgtgcagggtggtactgtttgtattcccggtgccttcggatgcggtaagactgtcatttctcagtctgtatccaagtcctccaacagtgatatcatcgtctacgttggttgtggtgagcgtggtaacgagatggctgaagtgttgatggacttccccgagctttcgatcgaaatcgatggtcgcaaagagcctatcatgaagcgtacatgtcttatcgccaatacatccaacatgcctgtcgccgcgcgtgaggcctccatttacaccggtatcaccatcgccgagtacttccgtgaccagggtaagaacgtggctatgatggccgattccagttctcgttgggccgaggcgcttcgtgaactttccggtcgtctgggagagatgcctgcagaccagggtttccccgcctacctgggtgccaagctcgcttccttctacgaacgtgctggaaagagtgtggcgctgggaagccctgagagaattggcagtgtcagtattgtcggtgccgtcagtccccctggtggtgatttctcagatcctgtcactactagtacccttggtatcgtccaggtgttctggggtctcgacaagaagctggcccagcgaaagcatttcccttccatcaacacttcgatgtcctacagcaagtacactaccgtcttggacaaattctacgagaagaactaccccgagttcccccgcctgcgtgaccagattcgtgagctgttgaccaagtccgaagaactggaccaggtcgtgcagctggtcggtaaggccgccctgggtgattctgataagatcgcattggatgtggctgccatggtgaaggatgatttccttcagcaaaacggatacagtgactacgatcagttctgccctctgtggaagacggaatacatgatgaaggccttcatgggctaccatgacgaagcgcagaaggctattgctcagggtcaaaactgggccaaggttcgcgaaccactgccgacatccagactgccctgcggaacatacagttgcgaggtcccggaaaaccaacaagaggtctcagagaagtacgagaaggttcttcagaccatgtccgagcgattcgcgtcggtgtcggatgagtaa

SEQ ID NO: 20 - DNA, 1776 bp, Saccharomyces cerevisiae
aagaatctcattagaagaccatgctgaatctgaatatggtgccatctattctgtctctggtccggtcgtcattgctgaaaatatgattggttgtgccatgtacgaattggtcaaggtcggtcacgataacctggtgggtgaagtcattagaattgacggtgacaaggccaccatccaagtttacgaagaaactgcaggccttacggtcggtgaccctgttttgagaacaggtaagcctctgtcggtagaattgggtcctggtctgatggaaaccatttacgatggtattcaaagacctttgaaagccattaaggaagaatcgcaatcgatttatatcccaagaggtattgacactccagctttggataggactatcaagtggcaatttactccgggaaagtttcaagtcggcgatcatatttccggtggtgatatttacggttccgtttttgagaattcgctaatttcaagccataagattcttttgccaccaagatcaagaggtacaatcacttggattgctccagctggtgagtacactttggatgagaagattttggaagttgaatttgatggcaagaagtctgatttcactctttaccatacttggcctgttcgtgttccaagaccagttactgaaaagttatctgccgactatcctttgttaacaggtcaaagagttttggatgctttgtttccttgtgttcaaggtggtacgacatgtattccaggtgcttttggttgtggtaagaccgttatctctcaatctttgtccaagtactccaattctgacgccattatctatgtcggctgcggagaaagaggtaatgaaatggcagaagtcttgatggaattcccagagttatatactgaaatgagcggtactaaagaaccaattatgaagcgtactactttggtcgctaatacatctaacatgccggttgcagccagagaagcttctatttacactggtatcactcttgcagaatacttcagagatcaaggtaaaaatgtttctatgattgcagactcttcttcaagatgggctgaagctttgagagaaatttctggtcgtttgggtgaaatgcctgctgatcaaggtttcccagcttatttgggtgctaagttggcctccttttacgaaagagccggtaaagctgttgctttaggttccccagatcgtactggttccgtttccatcgttgctgccgtttcgccagccggtggtgatttctcagatcctgttactactgctacattgggtatcactcaagtcttttggggtttagacaagaaattggctcaaagaaagcatttcccatctatcaacacatctgtttcttactccaaatacactaatgtcttgaacaagttttatgattccaattaccctgaatttcctgttttaagagatcgtatgaaggaaattctatcaaacgctgaagaattagaacaagttgttcaattagttggtaaatcggccttgtctgatagtgataagattactttggatgttgccactttaatcaaggaagatttcttgcaacaaaatggttactccacttatgatgctttctgtccaatttggaagacattcgatatgatgagagccttcatctcgtatcatgacgaagctcaaaaagctgttgctaatggtgccaactggtcaaaactagctgactctactggtgacgttaagcatgccgtttcttcatctaaattttttgaaccaagcaggggtgaaaaggaagtccatggcgaattcgaaaaattgtt

SEQ ID NO: 21 - DNA, 1782 bp, Saccharomyces pastorianus
aggaaataaaaagaatctcactggaagaccacgctgaatctgaatatggttccatctactctgtctctggtccggtcgtcattgctgaaaacatgattggctgtgccatgtacgaattggtcaaggtcggtcacgacaacctggtgggtgaagttattagaatcgacggtgataaagccaccattcaagtctatgaagaaactgctggtcttacggtcggtgaccctgttttgagaacaggtaagcctttgtcggtggaattgggtcctggtttgatggaaactatctatgacggtattcaaagacctttgaaagccattaaggaagaatcgcaatcgatttacattccaagaggtattgataccccatctttggacagaaccatcaaatggcaattcactccaggtaagttccaagtcggtgaccatatctctggtggtgacatttacggttccgttttcgaaaattccctgatttcgagccataagattcttttgccaccaagatctagaggtaccatcacctggattgctccagctggtgaatacactttggatgaaaaaattttggaagtcgaatttgacggcaagaaatctgatttcactctttaccacacttggccggtccgtgttccaagaccagtcaccgaaaaattatctgccgattatcctttgttgacaggccaaagagttttagacgctttgttcccttgtgttcaaggtggtacgacatgtattccaggtgcctttggttgtggtaagacagttatctctcagtccttatcaaagtactctaattctgacgctattatctacgtcggttgtggtgaacgtggtaatgaaatggcagaagtcttgatggaattccctgaattatacactgaaatgagtggtactaaagaaccaatcatgaagcgtactactttggttgctaacacatctaacatgcctgttgctgccagagaagcttccatatacactggtatcacacttgcagaatacttcagagatcaaggtaagaacgtctctatgattgctgactcttcttcaagatgggctgaagctttaagagaaatttctggtcgtttgggtgaaatgcctgctgatcaaggtttcccagcttatttgggtgctaaactagcttccttttatgaaagagctggtaaagctgttgctttgggttctccagatcgtattggttctgtttccattgttgctgctgtttctccagccggtggtgatttctcagatcctgttactacagctactttgggtatcactcaagtcttttggggtttggataagaaattggctcaaagaaagcatttcccatctatcaacacatctgtctcttactctaagtacaccactgttttgaacaagttttacgattccgattatcctgaattccctgtcttgagagatcgtatgaaggaaattttgtctaacgctgaagaattggaacaagtcgtccaattggttggtaagtctgctttatccgatagtgacaagattaccttggatgttgctgctttggttaaggaagatttcttacaacaaaatggttactctacctacgacgctttctgtccaatttggaagacctatgatatgatgagagcattcattgcatatcatgacgaagctcaaaaagctgttgctaatggtgccaactggtcaaaattagcagactctactagtgatgttaaacattccgtttcttcatctaaattttttgaaccaagtaggggtgaaaaagaagtgcatggcgatttcgaaaagt

SEQ ID NO: 22 - DNA, 1824 bp, Neurospora crassa
atggctccccaacaaaatggcgccgaggtggatggcatccataccggcaagatctactcggtctccggccccgtcgttgtcgccgaggatatgattggtgttgctatgtatgagttggttaaagtcggtcacgatcaactagttggtgaagtcattcgtatcaatggcgaccaagcaaccattcaagtatacgaagagacggctggtgtcatggttggcgatcccgtactacggacaggcaagcctctctctgtcgaacttggccctggtctcctgaacaacatctacgatggtatccagcgccccctcgagaagattgccgaggcttccaacagcatttacattccccgcggtattgccacccctgcgctggaccgcaagaagaaatgggagttcacaccgaccatgaaggttggcgatcacatcgcgggtggtgacgtctggggtactgtttacgagaactcgtttatctctgtccacaagattctcctccctccccgggcccgtggtaccatcactaggatcgccgagaagggcgagtacaccgttgaggagaagatcctcgaggtcgagttcgatggcaagaagaccgaatatcccatgatgcagacctggcctgtccgtgtaccgcgccctgcggccgagaagcattctgccaaccagcctttccttgtcggccagcgtgtgctcgacgctctcttcccctcggttcagggcggtactgttgctattcccggtgctttcggctgcggcaagactgtcatttctcagtccgtctccaagttctccaacagtgacgttatcgtatacgtcggttgtggtgagcgcggtaacgagatggctgaagtcttgaaggatttccccgagctgtctatcgaggtcgacggccgcaaggagcccatcatgaagcgcacgaccctcatcgccaacacctctaacatgcccgtcgccgctcgtgaggcctccatctacacgggtatcacagttgccgagtacttcagagatcagggcatgaacgttgccatgatggctgactcttcgtctcgttgggctgaggcgctcagagaaatttcgggtcgtctaggagaaatgccggctgatcagggtttccccgcttaccttggtgccaagctcgcctccttctacgaacgcgccggcaaggtccaagctcttggtagcccgccgcgcgagggtagtgttagcatcgttggtgctgtctctccccccggtggtgatttctctgatcccgtcacttctgccaccctcggtatcgtgcaggtcttctggggtctcgacaagaagcttgcacagcgcaagcacttcccgtccatcaacacctccgtcagttacagcaagtacctcaccattctcgacaagtggtatgagagggagtaccccgacttcccccgcctccgcgaccgcatccgccagctcctttccgacagcgaagagctcgaccaggtcgtccagctggttggcaagtcggcgctctcggatcccgacaagatcacgctcgacatggcgacgctcatcaaggaggacttcctccagcaaaacggctactcagactacgaccagttctgtcctatttggaagacggagtggatgatgaagctcatgatgggattccacgacgaggcacaaaaggcaattgctcagggccagaactggaacaaggtgcgcgaggccacccaggatctgcaagcccagttgaagagtctcaagttcgaggtgcctagtgaaggacaagagaagatttgcaagaagtacgaggcgatacagcagcagatgctggacaagttcgcgtctgtcattgatgagtga

SEQ ID NO: 23 - DNA, 1848 bp, Candida albicans
gccggagctttagaaaatgcaacaaaggaaattaaacgtctttcattagaagacacccatgaatcccaatatggacaaatctattctgtctcaggtccagttgtggttgcagaaaacatgattggatgtgcaatgtatgaattagtcaaagttggtcatgataatttggttggtgaagtcattagaattaatggcgataaggccactatccaagtttatgaagaaactgccggtgtcactgttggtgacccggttttaagaactggtaaaccattatctgttgagttgggtccagggttaatggaaactatttatgatggtattcaaagaccattgaaggcaattaaagatgaatcccaatccatttatattccaagaggtattgatgttccagctttatcaagaactactcaatatgatttcactcctggtaaattgaaagtaggtgaccatattactggtggtgatattttcgggtctatttacgaaaattctttgttagatgatcataaaatcttgttgccaccaagagctagagggactattacatctattgctgaatctggttcttacaatgttgaagatactgttttagaagttgaatttgatgggaaaaaacacaaatactccatgatgcacacctggccggtgagagttccaagaccagttgctgaaaaattaagtgctgattatccattgttgactggtcaaagagttttggattctttatttccatgtgtccaaggtggtaccacttgtattcctggtgcctttggttgtggtaaaactgttatttcacaatcattatcgaaattctccaattcagatgttattatttatgttggttgtggtgaacgtggtaatgagatggctgaagttttaatggaattcccagaattatacactgaaatatccggtagaaaagaaccaattatgaaacgtaccactttagttgccaacacatctaatatgccggtcgctgctagagaagcttctatttacactggtattactttagctgaatatttcagagatcaaggtaagaatgtgtctatgattgccgattcttcatcacgttgggccgaagcattgagagaaatctctggtagattgggagaaatgccggctgatcaaggtttccctgcttacttgggtgccaaattggcttctttctatgaacgtgctggtaaagccactgctttgggatctccagatagaattggttcagtttctattgttgctgctgtttcaccagctggtggtgatttctctgatccagttactactgccactttgggtattactcaagttttctggggtttagataaaaaattggctcaaagaaaacatttcccatctatcaacactagtgtttcatattccaaatacaccaatgttttgaacaaatattatgattccaattatccagaatttgctcaattgagagataaaattagagaaattttgtctaatgctgaagaattggaacaagttgtgcaattagttggtaaatctgctttatcagattccgataagattacattagatgttgccacattaatcaaagaagactttttacaacaaaatggttattcttcttatgatgcattctgtccaatttggaaaacttttgatatgatgagagcatttatttcatattatgatgaagcccaaaaagccgttgctaatggtgctcaatggtcaaaattagctgaaagtactagtgatgttaaacattctgtttcatcagctaaattttttgaaccatcaagaggtcaaaaagaaggtgaaaaagaatttagtgaattattatccactatttctgaaaggtttgccgaagcttctgaa

SEQ ID NO: 24 - DNA, 1878 bp, Sclerotinia sclerotiorum
atgtccagccaggaccaacagcagcaacaacagccggcgcaaacacagacttcgacgtcttcgagttctaataatgaaaacgctactacggcgacttcatcaattcagcaaaatgttgtagctgatgatagtttactatgtcagtgggagaaatgttcggaaagatgtcccactccagaagctttatttgatcacatctgcgagaaacatgttggaaggaagagcaccaataacttaaaccttacttgcggttggaattcatgccgtactactaccgtcaaacgcgatcatattacatctcacattcgtgttcatgtgccactgaaaccacataaatgtgaattctgtggaaaggcattcaagcgtccacaagatttgaagaaacatgtcaagacccatgccgatgattccgttttattgagaactccagaacaatctggtggttcaaatgggggatacagacaaccaggcggtaaagtaattgctaatttgcaacaccttgcagccaatcctatgggttattatgatcataatgcttcgatgcatcctggttctgccggggtttatggcaattctcatcacggtggtcatagtggatattatgcacctgcgcactctcaacaatcttcatatggaggaggcccaggctattatcaaatgtctcacaaccctgatctcggtcaacatgcagcttgggatgagaaaaagcgaaatttcgataacttgaatgatttcttcggtgccgctaagcgtcgtcaaattgatgctcattcttatcaacaagtcaatcagagattgatgcaattacaaggaattcctattggtacaggtggaggtatctctgattacattcattcagcacctcaattggttccaattgacggtcacggcggtcacggacatggagggcctatgccacaacaccaatactccctccctctcccgaacttgcggaccaagagtgatttgaattccattgatcaattcttggagcaaatgcaatcaacagtatacgaaagttcaaacgctgctgccgcagctggtattcaccaacctggtgctcactacactcaccaagctctcaacttccgtcaaagtcactcgccaccacaaactcatatccacaatattggttcaatggcgccacatgtttccacttcttacgcttcagcaccaatgaccgcaactcactcatctcattcagtttcttctggtacaccagctttaacacctccctcaagttccgtttcatatacttccggaaattccccgatgtcctcaagtggaatgtctcccatctcaagacacagttcaacatcaaatgcagcataccctaatcttccagctgtaacgcttggatactccccacatcattcagcaaccgcaccaacatccacacttggtacaaacttcgatagtgatccccgtcgtcgttactccggtggggtgctccaaagaagtgctggtggacttaactcaagtcaatatcgcgagtctatggagacatctaccgttggttctccaacaccatctccaaaggaaacaacacctcgccctgagtcaattgttaagacagaagttaccaataatattgacccagctctttctgatgctggttcaccttctgttcgatcagttgatactttggagagtgctcgtgatagagctgaagaggcatggattgaaaacattcgtgtcattgaagctttaagaagatatgtcagtgatcgtcttcaaaatggagagtatgtcaaggatgaagaagacgaagatgtatctatggccgacactgatatgcaagatacacaagtaaagactgaggagaagcctgtcgaaagtttgtatccagtgttgaaaacggatgatgatgatgagtag

SEQ ID NO: 25 - DNA, 17 bp, Artificial Sequence
  Description of Artificial Sequence Synthetic Primer
ttygaycayathtgyga

SEQ ID NO: 26 - DNA, 20 bp, Artificial Sequence
  Description of Artificial Sequence Synthetic Primer
tcytcrtcytcytcrtcytt

SEQ ID NO: 27 - DNA, 18 bp, Artificial Sequence
  Description of Artificial Sequence Synthetic Primer
ccatgcccarcaycarta

SEQ ID NO: 28 - DNA, 19 bp, Artificial Sequence
  Description of Artificial Sequence Synthetic Primer
ttytcdatccagcytcytc

SEQ ID NO: 29 - DNA, 3719 bp, Sclerotinia sclerotiorum
taacgacatctggcgagtaccactctttctttatctacttgcttgcatttcatggggaaacaatacccttcttttgcctactccggagtccgtacttactgtacgtaatgaagaatggactctacagtaccccgacgatgcagcctcctgcatcaatatatttctaatattaactatcctttgggttaaactttacctacgtttagatagtccaaagtatgtggcctcaacctttcctcccacctgcatcatgataaagttcacataaatgtcagcaattgtgccaatgctctctaggttaaacttctgacattgtgcaaatgctctctaggttaaactctcttttaaatcaatcttcggttcatacttgtatggtatcatttggaatctcatgatatgaaatgaatatgaaaccgttgtaatatgtgttaatggaaagaggtcagcaaatgtaaagaagagcaagcaaaagcaggaaaaacagcaaaacacagcccgaatgccaagagaatacgcagtgccaagagagccaagtttctccgatcttggcctcggggcgcaatctcttggatgtgaaagggcgaagaaaacgtattgccaatttctgcgacattgacaaaagaatctggtgcatgttccttccatacacgccaagacatttgaaccaattaaataatccactcaagtttgaaccgagtggaatgccaagagactaggagtggaagccaaacttaatttagtcgccaagagtttttgccaagacctagtcacgcccagtctttccgttattcttacaagaaggcaccaagatcctccctttggcttggagactcgtttgagaaacaatttcgaggaatacactttttatcactcacttcctttgattctagttttgcgtcaacaatcttaacagaacttttggacattccatcaatcaatcaatcaaattcattcaacttctaatattgatagaaggaacagacccataactttttactagtctatcttcttcttccacacacagtttagtctgtgaatagatcattttagccgacaattgattattggttaattccaaccaccttatccatacacccaaaatgtccagccaggaccaacagcagcaacaacagccggcgcaaacacagacttcgacgtcttcgagttctaataatgaaaacgctactacggcgacttcatcaattcagcaaaatgttgtagctgatgatagtttactatgtcagtgggagaaatgttcggaaagatgtcccactccagaagctttatttgtaggttgattttacctcagcactcatatcttcaaacaccatctcaatccaaagtccttgtactaacatgtaatttccaggatcacatctgcgagaaacatgttggaaggaagagcaccaataacttaaaccttacttgcggttggaattcatgccgtactactaccgtcaaacgcgatcatattacatctcacattcgtgttcatgtgccactgaaaccacataaatgtgaattctgtggaaaggcattcaagcgtccacaagatttgaagaaacatgtcaaggtacgagcaagagctggtgtgtattcctttcaccctgctaacaatcacccttagacccatgccgatgattccgttttattgagaactccagaacaatctggtggttcaaatgggggatacagacaaccaggcggtaaaggtacgtaagggcgcaagttgcctttgttcttatatatgtctaacatttttcttatgtagtaattgctaatttgcaacaccttgcagccaatcctatgggttattatgatcataatgcttcgatgcatcctggttctgccggggtttatggcaattctcatcacggtggtcatagtggatattatgcacctgcgcactctcaacaatcttcatatggaggaggcccaggctattatcaaatgtctcacaaccctgatctcggtcaacatgcagcttgggatgagaaaaagcgaaatttcgataacttgaatgatttcttcggtgccgctaagcgtcgtcaaattgatgctcattcttatcaacaagtcaatcagagattgatgcaattacaaggaattcctattggtacaggtggaggtatctctgattacattcattcagcacctcaattggttccaattgacggtcacggcggtcacggacatggagggcctatgccacaacaccaatactccctccctctcccgaacttgcggaccaagagtgatttgaattccattgatcaattcttggagcaaatgcaatcaacagtatacgaaagttcaaacgctgctgccgcagctggtattcaccaacctggtgctcactacactcaccaagctctcaacttccgtcaaagtcactcgccaccacaaactcatatccacaatattggttcaatggcgccacatgtttccacttcttacgcttcagcaccaatgaccgcaactcactcatctcattcagtttcttctggtacaccagctttaacacctccctcaagttccgtttcatatacttccggaaattccccgatgtcctcaagtggaatgtctcccatctcaagacacagttcaacatcaaatgcagcataccctaatcttccagctgtaacgcttggatactccccacatcattcagcaaccgcaccaacatccacacttggtacaaacttcgatagtgatccccgtcgtcgttactccggtggggtgctccaaagaagtgctggtggacttaactcaagtcaatatcgcgagtctatggagacatctaccgttggttctccaacaccatctccaaaggaaacaacacctcgccctgagtcaattgttaagacagaagttaccaataatattgacccagctctttctgatgctggttcaccttctgttcgatcagttgatactttggagagtgctcgtgatagagctgaagaggcatggattgaaaacattcgtgtcattgaagctttaagaagatatgtcagtgatcgtcttcaaaatggagagtatgtcaaggatgaagaagacgaagatgtatctatggccgacactgatatgcaagatacacaagtaaagactgaggagaagcctgtcgaaagtttgtatccagtgttgaaaacggatgatgatgatgagtagttgttaattttaagcttcatgaagttgattgaaatgggcggagttttgcttgcttggtataccaagggtggattgaatggatttatacattatttgataggagtggatggatggcgttggtatggatggagtatgaattttaacctatctactttgatcgaggaatgagggagaatggtgtgcagtaataccttcgttttattgaatggaatgacctgtcattaagtggatgatagtatatgtatacaataagcaagatttcgctttttctctatatcatctccatatggtgttttatgatgtgattcggtttgatgaatattttgtgggaagagatgacatgtaatctactcgggcccgagaagagccccgcacatatgtgagcattgctttaacaactaccccactttaaatatacctctttattccatcgtatctctattattgaatatttatttatttatctatctgtctacctatctatcttttcgttataaacctccctcgttttccacacacaataacacctaccatccatcaaatctcga

SEQ ID NO: 30 - DNA, 9668 bp, Sclerotinia sclerotiorum
tcgacgcggcggccgcagactcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttgagctcgcgactagaccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggactagtgcgcccgggataggccggccgcggtgtctcgcacacggcttcgacggcgtttctggcgcgtttgcagggccatagacggccgccagcccagcggcgagggcaaccagcccggtgagcgtcggaaagggtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggtcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagatgctgcagactagggcgagatctaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcttctagggcggtcgacgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaaggcttctagtttttctctatccaggcttcttcagctctatcacgagcactctccaaagtatcaactgatcgaacagaaggtgaaccagcatcagaaagagctgggtcaatattattggtaacttctgtcttaacaattgactcagggcgaggtgttgtttcctttggagatggtgttggagaaccaacggtagatgtctccatagactcgcgatattgacttgagttaagtccaccagcacttctttggagcaccccaccggagtaacgacgacggggatcactatcgaagtttgtaccaagtgtggatgttggtgcggttsctgaatgatgtggggagtatccaagcgttacagctggaagattagggtatgctgcatttgatgttgaactgtgtcttgagatgggagacattccacttgaggacatcggggaatttccggaagtatatgaaacggaacttgagggaggtgttaaagctggtgtaccagaagaaactgaatgagatgagtgagttgcggtcattggtgctgaagcgtaagaagtggaaacatgtggcgccattgaaccaatattgtggatatgagtttgtggtggcgagtgactttgacggaagttgagagcttggtgagtgtagtgagcaccaggttggtgaataccagctgcggcagcagcgtttgaactttcatatactgttgattgcattcgctccaagaattgatcaatggaatacaaatcactcttggtccgcaagttcgggagagggagggagtattggtgttgcggcatcggcctagtaacggccgccagtgtgctggaattcgcccttccgatgccgcaacaccaatactccctccctctcccgaacttgcggaccaagagtgatttgtattccattgatcaattcttggagcgaatgcaatcaacagtatatgaaagttcaaacgctgctgccgcagctggtattcaccaacctggtgctcactacactcaccaagctctcaacttccgtcaaagtcactcgccaccacaaactcatatccacaatattggttcaatggcgccacatgtttccacttcttacgcttcagcaccaatgaccgcaactcactcatctcattcagtttcttctggtacaccagctttaacacctccctcaagttccgtttcatatacttccggaaattccccgatgtcctcaagtggaatgtctcccatctcaagacacagttcaacatcaaatgcagcataccctaatcttccagctgtaacgcttggatactccccacatcattcagsaaccgcaccaacatccacacttggtacaaacttcgatagtgatccccgtcgtcgttactccggtggggtgctccaaagaagtgctggtggacttaactcaagtcaatatcgcgagtctatggagacatctaccgttggttctccaacaccatctccaaaggaaacaacacctcgccctgagtcaattgttaagacagaagttaccaataatattgacccagctctttctgatgctggttcaccttctgttcgatcagttgatactttggagagtgctcgtgatagagctgaagaagcctggatagagaaaagggcgaattctgcagatatccatcacactggcggccgctcgagcatgcatctagaactagtggatctgctagagtcagcttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggacctgcagaagctttatagatcttatatgagctcgatcatgagcggagaattaagggagtcacgttatgacccccgccgatgacgcgggacaagccgttttacgtttggaactgacagaaccgcaacgattgaaggagccactsagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcagatcagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagcccgggtatg

SEQ ID NO: 31 - DNA, 9833 bp, Sclerotinia sclerotiorum
tcgacgcggcggccgcagactcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttgagctcgcgactagaccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggactagtgcgcccgggataggccggccgcggtgtctcgcacacggcttcgacggcgtttctggcgcgtttgcagggccatagacggccgccagcccagcggcgagggcaaccagcccggtgagcgtcggaaagggtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggtcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagatgctgcagactagggcgagatctaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcttctagggcggtcgacgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaaggcttctagttccatttcgtccatgccctcaccagtgtaccaatgcaagaaagcctttcttctgaacatagcagtgaattgatcaccgacacgcttgaagagttcttggatggaggtcgagttaccgacgaaggtggaggacatcttgagaccacggggaggaatggagcaaagggcggtttggacattgttagggatccactcgacgaagtaggaagagttcttgttttggacattgcgcatttggtcctcaacctccttcatggaaaccttaccacggaagatagcagagcaggttaagtaacgaccgttacggaaatcggaagcggccatcatgttcttaggatcatacatttgttgggtcaactctggaacagtaacagcacggaaagagtgtgcgccacgactggtcaaaggagcaaatccaaccatgaagaaatgaagacgggggaatggaaccatgttgacagccaactttcggagatctgagttaagttgaccagggaaacggagacaggtggtaacaccggacatgacagcggagaccaagtggttaagatctccgtaggatgggtggctgagcttcaaggttctcatgcaaatgtcgtagagagcctcgttgtcgatacagaaggtctcgtcagagttctcgaccaattgatgaacagagagagtagcgttatatggctcgacgacggtatcggaaacctttggcgatgggacgacggagaaggtagccatcatacgatctgggaactcctcacggatcttggaaatcaaaagcgtacccataccggcaccagttccaccaccgagagagtgggtgatttggaaaccttgaaggcagtcacagccctcagcctcacgacgaacgacatcaagaacttggtcgaccaagttcttgatgtcgttcgtcgtgaggctgagggctgtgactgccttcaaggtttccaaatcacccactctctcggtggtggaactggtgccggtatgggtacgcttttgatttccaagatccgtgaggagttcccagatcgtatgatggctaccttctccgtcgtcccatcgccaaaggtttccgataccgtcgtcgagccatataacgctactctctctgttcatcaattggtcgagaactctgacgagaccttctgtatcgacaacgaggctctctacgacatttgcatgagaaccttgaagctcagccacccatcctacggagatcttaaccacttggtctccgctgtcatgtccggtgttaccacctgtctccgtttccctggtcaacttaactcagatctccgaaagttggctgtcaacatggttccattcccccgtcttcatttcttcatggttggatttgctcctttgaccagtcgtggcgcacactctttccgtgctgttactgttccagagttgacccaacaaatgtatgatcctaagaacatgatggccgcttccgatttccgtaacggtcgttacttaacctgctctgctatcttccgtggtaaggtttccatgaaggaggttgaggaccaaatgcgcaatgtccaaaacaagaactcttcctacttcgtcgagtggatccctaacaatgtccaaaccgccctttgctccattcctccccgtggtctcaagatgtcctccaccttcgtcggtaactcgacctccatccaagaactcttcaagcgtgtcggtgatcaattcactgctatgttcagaagaaaggctttcttgcattggtacactggtgagggcatggacgaaatggaaagggcgaattctgcagatatccatcacactggcggccgctcgagcatgcatctagaactagtggatctgctagagtcagcttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggacctgcagaagctttatagatcttatatgagctcgatcatgagcggagaattaagggagtcacgttatgacccccgccgatgacgcgggacaagccgttttacgtttggaactgacagaaccgcaacgattgaaggagccactsagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcagatcagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagcccgggtatg

SEQ ID NO: 32 - DNA, 9762 bp, Sclerotinia sclerotiorum
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagcccgggtatgtcgacgcggcggccgcagactcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttgagctcgcgactagaccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggactagtgcgcccgggataggccggccgcggtgtctcgcacacggcttcgacggcgtttctggcgcgtttgcagggccatagacggccgccagcccagcggcgagggcaaccagcccggtgagcgtcggaaagggtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggtcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagatgctgcagactagggcgagatctaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcttctagggcggtcgacgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaaggcttctagagcaaaggaatccgagagatatattgacaagactaaacaccctcttacactcttccaaaacgactcttcactatcctcccttccgcggcaatttcatattcatgcatcaatcaagttctccacttgcgcatccttcgccttaccttcctcactgttatccttggtatccttgttgccagcacgtcctttacccttgcggtcagctgcagatcgttggtagaattcagccaaaaccttttgtgggatacgattgagcaattccttggggtaaatacgaagtaagctccatgcttgatcgagtgactcataaatggttctcgattcgtaagcagattgtgaaatgaaagtacgctcgaatttctccaaaaattccaacgagagcttgtcctcagaagataaagcttcctcgccaacaactgccttcatggatgctgcatcacgaccaatggcatatttggcgtacaattggttggaaacatcaccgtgatcctttctggtgagcttttctccaatagcagacttcatgagacgggagagagatggcagcacgttgataggtgggtagataccacggttgtgtagttgacgatcgatgaaaatttgtccttcagtaatataaccagtcaaatcgggaatagggtgagtaatatcgtcgttaggcatagtcaagataggaatttgagtaatggatccatttcgaccttggacacgaccagcacgttcataaatggtggacaaatccgtgtacatataaccggggtatccacgacgtccaggtacttcttcacgggcagctgaaacctcacgaagagcatcacagtatgcagtcaaatcggtaagaataaccaaacaatgcttctccaattggtcctaggaccaattggagaagcattgtttggttattcttaccgatttgactgcatactgtgatgctcttcgtgaggtttcagctgcccgtgaagaagtacctggacgtcgtggataccccggttatatgtacacggatttgtccaccatttatgaacgtgctggtcgtgtccaaggtcgaaatggatccattactcaaattcctatcttgactatgcctaacgacgatattactcaccctattcccgatttgactggttatattactgaaggacaaattttcatcgatcgtcaactacacaaccgtggtatctacccacctatcaacgtgctgccatctctctcccgtctcatgaagtctgctattggagaaaagctcaccagaaaggatcacggtgatgtttccaaccaattgtacgccaaatatgccattggtcgtgatgcagcatccatgaaggcagttgttggcgaggaagctttatcttctgaggacaagctctcgttggaatttttggagaaattcgagcgtactttcatttcacaatctgcttacgaatcgagaaccatttatgagtcactcgatcaagcatggagcttacttcgtatttaccccaaggaattgctcaatcgtatcccacaaaaggttttggctgaattctaccaacgatctgcagctgaccgcaagggtaaaggacgtgctggcaacaaggataccaaggataacagtgaggaaggtaaggcgaaggatgcgcaagtggagaacttgattgatgcatgaatatgaaattgccgcggaagggaggatagtgaagagtcgttttggaagagtgtaagagggtgtttagtcttgtcaatatatctctcggattcctttgctctagaactagtggatctgctagagtcagcttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggacctgcagaagctttatagatcttatatgagctcgatcatgagcggagaattaagggagtcacgttatgacccccgccgatgacgcgggacaagccgttttacgtttggaactgacagaaccgcaacgattgaaggagccactsagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcagatcagatcaagagacaggatgaggatcgtttcgc

SEQ ID NO: 33 - DNA, 501 bp, Phakopsora pachyrizi
cgtaccgtctcggctgtcaatggtcctctggtggtattagacaatgttcattttccttcttacaatgagattgtgatgctcaccttaccagatggaacccagaggggtggccaagtcttggaggtcaatggcaagaaggcgatcgtccaagtgtttgaaggaacttctgggatcgatgtgagagctacccatgtggagttcagtggatcgagtatgaaactaccggtcagtgaggacatgttaggtaggatctttaacggatctggtaaaccgatcgataaaggaccaaaggtatttgcagaagactacttggatatagatggtggctctcccataaacagaatttaccctgaagaaatgattcagactggtatatctaccattgatgcaatgaactcaattgcaagaggacaaaagattccaatattttctgcatctggtctacctcacaacgagatcgccgctcagatctgcaggcaagctggactggttaacaaggga

SEQ ID NO: 34 - DNA, 486 bp, Phakopsora pachyrizi
gaattcgatcgagcaacgatccaggtctacgaggagaccagtgggatgactataggtgatcctgtgctgaggacgggtaaacctttgagtgttgagttgggaccgggtctgatgaacatctatgatgggattcaaagacctctaaagtcgatctctgaactctcaaactcgatctacatacccagaggtatcaacacacaggcccttgacagaagccagagctgggaattcactccgactaattacaaaattggtgatcatctgagtggtggtgatatctatggcacagtttacgaaaactcactcgtctctgctcacaagatcatgcttccaccaagggcgatgggtacaatcactcaaattgctgatcgatcgaaagggcaagaatctcagcacacgatgtgccagctctggcccgttagagctcctcggccagttaccgagaaactcacacccgattttccactattgactggtcaaagagtt

SEQ ID NO: 35 - DNA, 819 bp, Phakopsora pachyrizi
aggatatgggggatgaacattgcgatgatggccgattctacgtctcgttgggctgaggctcttcgtgaattttctggtcgactggcagagatgccggcagactctggttatcccgcttatctgggtaccaaacttgctagtttttacgagagagctggtaagacgaggtgtggctcggaacagtctcgatcattggttctccaatgcagagtatcacctcctggtggagacttttcggatcccgttacatctcaaacattgacgattgctcaagtgttttggggtttggacaagaagctggctgttaactggaacgtgtcatactcaaagatttcttcttgtagatatgtctccgttcttcaaccgtggtatgcaaagactgagccggagtttgttaactaccgaaacaaagcaaaagatgtgctgcagaaggaggatgagcttgctgagattgtccagttggtcggtaaaagtgcattaggagaaggagacaaggtctgtggggacactttggatgttgccaggctggtaaaggatgattacttgcagcaaaacggaatgagtacatatgatagatactgccccttttacaagacgaatgcgatgctaaaaaacttgatgacctactacacggaggcacagaaggctgtcgaaaccaacacgggaggaaagagcttgagctgggcaaaggttagagatttaacaggtgatgagtggtacagattaagtcagatgaagttcgaggaccctaaggatggagaagaaagtctgatgaagaagtttaacgaagatatcatcaaaaagtttcagagcgtttca

SEQ ID NO: 36 - protein, 7 aa, Artificial Sequence
  Description of Artificial Sequence Synthetic Peptide
NNWAKGH

SEQ ID NO: 37 - protein, 7 aa, Artificial Sequence
  Description of Artificial Sequence Synthetic Peptide
EGMDEME

SEQ ID NO: 38 - protein, 7 aa, Artificial Sequence
  Description of Artificial Sequence Synthetic Peptide
IQVYEET

SEQ ID NO: 39 - protein, 7 aa, Artificial Sequence
  Description of Artificial Sequence Synthetic Peptide
EMPADQG

SEQ ID NO: 40 - protein, 6 aa, Artificial Sequence
  Description of Artificial Sequence Synthetic Peptide
FDHICE

SEQ ID NO: 41 - protein, 7 aa, Artificial Sequence
  Description of Artificial Sequence Synthetic Peptide
KDEEDED

SEQ ID NO: 42 - protein, 7 aa, Artificial Sequence
  Description of Artificial Sequence Synthetic Peptide
PMPQHQY

SEQ ID NO: 43 - protein, 7 aa, Artificial Sequence
  Description of Artificial Sequence Synthetic Peptide
EEAWIEN

(19) United States
(12) Patent Application Publication
(10) Pub. No.: US 2011/0061128 A1
(43) Pub. Date: Mar. 10, 2011

(54) IN PLANTA RNAi CONTROL OF FUNGI

(76) Inventors: James K. Roberts, Chesterfield, MO (US); John W. Pitkin, Wildwood, MO (US); Thomas H. Adams, St. Louis, MO (US)

(21) Appl. No.: 12/850,544

(22) Filed: Aug. 4, 2010

Related U.S. Application Data
(63) Continuation of application No. 11/670,409, filed on Feb. 1, 2007, now abandoned.
(60) Provisional application No. 60/765,112, filed on Feb. 3, 2006.

(51) Int. Cl. C12N 15/82; C07H 21/00; C07H 21/02; C12N 1/00; C12N 5/10; A01H 5/00; A01H 5/10; A01N 57/16; C07K 1/00; C11B 1/00; C12Q 1/68; A01P 3/00; A23L 1/00; A23K 1/00

(52) U.S. Cl. 800/279; 536/24.5; 435/243; 435/419; 800/301; 514/44 A; 530/370; 554/8; 435/6; 426/665

(57) ABSTRACT

The present invention relates to control of fungal and oomycete plant pathogens by inhibiting one or more biological functions. The invention provides methods and compositions for such control. By feeding one or more recombinant double stranded RNA molecules provided by the invention to the pathogen, a reduction in disease may be obtained through suppression of gene expression. The invention is also directed to methods for making transgenic plants that express the double stranded RNA molecules, and to particular combinations of transgenic agents for use in protecting plants from pathogen infection.

This application claims the priority of U.S. Provisional Patent Application 60/765,112, filed Feb. 3, 2006, the disclosure of which is incorporated herein by reference in its entirety.

### BACKGROUND OF THE INVENTION

1. Field of the Invention

The present invention relates generally to genetic control of plant disease. More specifically, the present invention relates to recombinant DNA technologies for post-transcriptionally repressing or inhibiting expression of target coding sequences in the cell of a fungal plant pathogen or host to provide a protective effect.

2. Description of Related Art

Plants are subject to multiple potential disease causing agents in the environment. Plant pathogens include various fungi, bacteria, viruses, nematodes, and algae, among others. A multitude of means have been utilized for attempting to control infection and disease by these pathogens. Compositions and agents for controlling infestations by pests such as bacteria, fungi, nematodes and viruses have been provided in the form of antibiotic compositions, antifungal compositions, nematocides, and antiviral compositions. Biological and cultural control methods have been attempted in numerous instances. Chemical compositions have typically been applied to surfaces on which pathogens are present or administered to pathogenic microorganisms in the form of pellets, powders, tablets, pastes, or capsules and the like, but the level of specificity of these compositions toward target organisms has often been less than desirable. Thus, there is a great need in the art for improvement of these methods and particularly for methods that would benefit the environment relative to the prior techniques.

Commercial crops and agroecosystems are often the targets of attack by pathogens. Substantial progress has been made in the last few decades towards developing more efficient methods and compositions for controlling plant pathogenic microorganisms, including chemical agents that have often been very effective in eradicating infectious agents. However, there are several disadvantages to using chemical agents. Chemical agents are not selective. Applications of chemical pesticides intended to control pathogens that are harmful to various crops and other plants exert their effects on non-target organisms as well, often effectively disrupting populations of beneficial microorganisms as well, for a period of time following application of the agent. Chemical agents may persist in the environment and often are slow to be metabolized, if at all. They may accumulate in the food chain, and particularly in the higher predator species. Repeated application of these chemical pesticidal agents may lead to the development of pathogen populations resistant to the agents. Accumulation of these chemical agents in species higher up the evolutionary ladder can also often occur. These agents may act as mutagens and/or carcinogens to cause irreversible and deleterious genetic modifications. Thus there has been a particularly long felt need for environmentally friendly methods for controlling or eradicating pathogen infestation on or in plants, i.e., methods that are selective, environmentally inert, non-persistent, and biodegradable, and that fit well into disease management schemes.

Antisense methods and compositions have been reported in the art and are believed to exert their effects through the synthesis of a single-stranded RNA molecule that in theory hybridizes in vivo to a substantially complementary sense strand RNA molecule. Antisense technology has been difficult to employ in many systems for three principal reasons. First, the antisense sequence expressed in the transformed cell is unstable. Second, the instability of the antisense sequence expressed in the transformed cell concomitantly creates difficulty in delivery of the sequence to a host, cell type, or biological system remote from the transgenic cell. Third, the difficulties encountered with instability and delivery of the antisense sequence create difficulties in attempting to provide a dose within the recombinant cell expressing the anti sense sequence that can effectively modulate the level of expression of the target sense nucleotide sequence.

Double stranded RNA mediated inhibition of specific genes in various organisms has been previously demonstrated. dsRNA mediated approaches to genetic control have been tested in the fruit fly Drosophila melanogaster (Kennerdell Cell 95:1017-1026). Kennerdell et. al. describe a method for delivery of dsRNA involving generating transgenic insects that express double stranded RNA molecules or injecting dsRNA solutions into the insect body or within the egg sac prior to or during embryonic development. Research investigators have previously demonstrated that double stranded RNA mediated gene suppression can be achieved in nematodes either by feeding or by soaking the nematodes in solutions containing double stranded or small interfering RNA molecules and by injection of the dsRNA molecules. Rajagopal et. al. (2002) described failed attempts to suppress an endogenous gene in larvae of the insect pest Spodoptera litura by feeding or by soaking neonate larvae in solutions containing dsRNA specific for the target gene, but was successful in suppression after larvae were injected with dsRNA into the hemolymph of 5^{th} instar larvae using a microapplicator. Similarly, U.S. Patent App. Pub. No. 2003/0150017 prophetically describes a preferred locus for inhibition of the lepidopteran larvae Helicoverpa armigera using dsRNA delivered to the larvae by ingestion of a plant transformed to produce the dsRNA. Development of plant diseases, for instance viral diseases, are also reported to have been suppressed by RNAi approaches in plant cells (e.g. Lindbo & Dougherty, 2005).

To date, no published information exists on RNAi-mediated gene suppression in fungi where the double-stranded (dsRNA) or small interfering (siRNA) molecules are taken up from artificial growth media (in vitro) or from plant tissue (in planta). The literature contains examples of RNAi-mediated gene suppression via transformation of DNA constructs into fungal cells either treated by cell wall alterations or electroporation; in other words the typical DNA transformation protocols used in fungi for the past 20 years (Chicas, Cogoni, and Macino; Cottrell and Doering; Mouyna et al.; Raponi and Arndt; Reese and Doering; Kadotani). Suppression of fungal infection of barley by interfering with expression of a plant gene via RNAi-mediated gene suppression has also been reported (Schultheiss et al.). The lack of RNAi-mediated gene suppression via fungal uptake of dsRNA molecules might have been due to degradation of the RNA outside of the cell or an inherent inability of fungal cells to take up dsRNA from the environment.

Biotrophic fungi possess various strategies to access host nutrients. Some utilize extracellular growth; some use intercellular growth; other grow largely intercellularly, but with specialized hyphae (haustoria) that grow into plant cell apoplasts. Finally, some may grow intracellularly, during at least part of their lifecycle. In each of these cases, host responses to fungal infection are suppressed (Mendgen and Hahn, 2002).

It has previously been impractical to provide dsRNA molecules for control of fungal plant pathogens. Therefore, there has existed a need for improved methods of modulating gene expression by repressing, delaying or otherwise reducing gene expression within a particular fungal pathogen for the purpose of controlling pathogen infestation or to introduce novel phenotypic traits.

### SUMMARY OF THE INVENTION

In one aspect, the invention provides a method of inhibiting expression of a target gene in a phytopathogenic microorganism. In certain embodiments, the method comprises modulating or inhibiting expression of one or more target genes in a phytopathogen that causes cessation of infection, growth, development, and/or reproduction, and eventually results in the death of the organism. The method comprises introduction of partial or fully, stabilized double-stranded RNA (dsRNA), including its modified forms such as small interfering RNA (siRNA) sequences, to the target phytopathogen, wherein the dsRNA inhibits expression of at least one or more target genes of the phytopathogen and wherein the inhibition exerts a deleterious effect upon the pathogen. The methods and associated compositions may be used for limiting or eliminating infection of a plant or plant cell by a phytopathogen, such as a fungus, in or on any host tissue or environment in which a pathogen is present by providing one or more compositions comprising the dsRNA molecules described herein in the host of the pathogen. The method will find particular benefit for protecting plants from fungal attack. In one embodiment, the pathogen is defined as a biotroph. In other embodiments, the pathogen is a necrotroph or a hemibiotroph. In a preferred embodiment, the pathogen is a fungus. The pathogen in particular may be a rust fungus, and may be the causal agent of Asian Soy Rust (e.g. Phakopsora pachyrizi).

In another aspect, the present invention provides exemplary nucleic acid compositions that are homologous to at least a portion of one or more native nucleic acid sequences in a target plant pathogenic microorganism. Specific examples of such nucleic acids provided by the invention are given in the attached sequence listing as SEQ ID NO:3-15; SEQ ID NO:18-23; SEQ ID NO:29; and SEQ ID NO:33-35.

In another aspect, the invention provides a method for designing and producing a nucleic acid molecule that is taken up in vitro or in planta by a plant pathogenic fungus in a form effective to allow for sequence specific suppression of fungal gene expression by an RNAi-mediated mechanism. In one embodiment, such a nucleic acid molecule is partially double stranded and is resistant to degradation by ribonuclease. In another embodiment, the nucleic acid is a siRNA. In another embodiment, the nucleic acid suppresses expression of a gene necessary for fungal growth. In yet another embodiment, the nucleic acid suppresses expression of a gene necessary for infection of host tissue by a fungus. In another embodiment, the nucleic acid suppresses expression of a gene necessary for fungal reproduction. In yet another embodiment, the nucleic acid suppresses expression of a gene necessary for uptake of nutrients by a fungal cell.

In another embodiment, the invention provides a method for modulating expression of a target gene in a fungal cell, the method comprising: (a) transforming a plant cell with a vector comprising a nucleic acid sequence encoding a dsRNA operatively linked to a promoter and a transcription termination sequence; (b) culturing the transformed plant cell under conditions sufficient to allow for development of a plant cell culture comprising a plurality of transformed plant cells; (c) selecting for transformed plant cells that have integrated the vector into their genomes; (d) screening the transformed plant cells for expression of the dsRNA encoded by the vector; (e) selecting a plant cell that expresses the dsRNA; (f) optionally regenerating a plant from the plant cell that expresses the dsRNA; whereby expression of the gene in the plant is sufficient to modulate the expression of a target gene in a fungal cell that contacts the transformed plant or plant cell. Modulation of gene expression may include partial or complete suppression of such expression.

In yet another aspect, the invention provides a method for suppressing a gene expressed in a plant pathogen, such as a fungus or oomycete, that comprises the step of providing in the tissue of the host of the pathogen a gene suppressive amount of at least one dsRNA molecule transcribed from a nucleotide sequence as described herein, at least one segment of which is complementary to an mRNA sequence within the cells of the pathogen. The method may further comprise observing the death or growth inhibition, of the pathogen, and the degree of host symptomatology. A dsRNA molecule, including its modified form such as an siRNA molecule, taken up by a pathogenic microorganism in accordance with the invention may be at least from about 80, 95, 96, 97, 98, 99, or about 100% identical to a segment of a RNA molecule transcribed from a nucleotide sequence selected from the group consisting of SEQ ID NO:3-15; SEQ ID NO:18-23; SEQ ID NO:29; and SEQ ID NO:33-35. In specific embodiments, such a sequence may be defined as having the aforementioned identity to (a) two or more segments of a single gene, (b) one or more segments of different genes, or (c) two or more segments of two or more genes.

In another embodiment, the invention provides a nucleic acid that suppresses expression of a host plant gene that is necessary for establishment or maintenance of a fungal infection, or development of plant disease symptoms.

Accordingly, in another aspect of the present invention, a set of isolated and purified nucleotide sequences as set forth in SEQ ID NO:3-15; SEQ ID NO:18-23; SEQ ID NO:29; and SEQ ID NO:33-35 is provided. The present invention provides a stabilized dsRNA molecule for the expression of one or more miRNAs for inhibition of expression of a target gene in a phytopathogenic microorganism, expressed from these sequences and fragments thereof. A stabilized dsRNA, including a miRNA or siRNA molecule can comprise at least two coding sequences that are arranged in a sense and an antisense orientation relative to at least one promoter, wherein the nucleotide sequence that comprises a sense strand and an antisense strand are linked or connected by a spacer sequence of at least from about five to about one thousand nucleotides, wherein the sense strand and the antisense strand may be a different length, and wherein each of the two coding sequences shares at least 80% sequence identity, at least 90%, at least 95%, at least 98%, or 100% sequence identity, to any one or more nucleotide sequence(s) set forth in set forth in SEQ ID NO:3-15; SEQ ID NO:18-23; SEQ ID NO:29; and SEQ ID NO:33-35. Such sequences may be defined as substantially perfectly complementary along the length of at least the shorter of the two strands.

The invention also provides one or more stabilization sequences, or “clamps”, which may be unrelated to the gene of interest. A clamp preferably comprises a GC rich region that serves to thermodynamically stabilize the dsRNA molecule, and may increase gene silencing.

Further provided by the invention is a fragment of a nucleic acid sequence selected from the group consisting of SEQ ID NO:3-15; SEQ ID NO:18-23; SEQ ID NO:29; and SEQ ID NO:33-35. The fragment may be defined as causing the death, growth inhibition, or cessation of infection by a plant pathogenic microorganism, when expressed as a dsRNA and provided to the microorganism. The fragment may, for example, comprise at least about 19, 21, 23, 25, 40, 50, 60, 80, 100, 125, 200, 400 or more contiguous nucleotides of any one or more of the sequences in SEQ ID NO:3-15; SEQ ID NO:18-23; SEQ ID NO:29; and SEQ ID NO:33-35, or a complement thereof, including the full length thereof. One beneficial DNA segment for use in the present invention is at least from about 19 to about 23, or about 23 to about 100 nucleotides, but less than about 2000 nucleotides, in length. Particularly useful will be dsRNA sequences including about 23 to about 300 nucleotides homologous to a phytopathogen target sequence. The invention also provides a ribonucleic acid expressed from any of such sequences including a dsRNA. A sequence selected for use in expression of a gene suppression agent can be constructed from a single sequence derived from one or more target pathogen species and intended for use in expression of an RNA that functions in the suppression of a single gene or gene family in the one or more target pathogens, or that the DNA sequence can be constructed as a chimera from a plurality of DNA sequences.

In yet another aspect, the invention provides recombinant DNA constructs comprising a nucleic acid molecule encoding a dsRNA molecule described herein. The dsRNA may be formed by transcription of one strand of the dsRNA molecule from a nucleotide sequence which is at least from about 80% to about 100% identical to a nucleotide sequence selected from the group consisting of SEQ ID NO:3-15; SEQ ID NO:18-23; SEQ ID NO:29; and SEQ ID NO:33-35. Such recombinant DNA constructs may be defined as producing dsRNA molecules capable of inhibiting the expression of endogenous target gene(s) in a plant pathogen cell upon being taken up. The construct may comprise a nucleotide sequence of the invention operably linked to a promoter sequence that functions in the host plant cell. Such a promoter may be tissue-specific and may, for example, be specific to a tissue type which is the subject of pathogen attack. In the case of a root or foliar pathogen, respectively for example, it may be desired to use a promoter providing root or leaf-preferred expression, respectively. In one embodiment, a pathogen inducible promoter may be used, for example, a promoter induced in a plant in response to a fungal or oomycete infection.

Nucleic acid constructs in accordance with the invention may comprise at least one non-naturally occurring nucleotide sequence that can be transcribed into a single stranded RNA capable of forming a dsRNA molecule in vivo through intermolecular hybridization. Such dsRNA sequences self assemble and can be provided in the nutrition source of a plant pathogenic microorganism to achieve the desired inhibition.

A recombinant DNA construct may comprise two different non-naturally occurring sequences which, when expressed in vivo as dsRNA sequences and provided in the tissues of the host plant of a plant pathogenic microorganism, inhibit the expression of at least two different target genes in the plant pathogenic microorganism. In certain embodiments, at least 3, 4, 5, 6, 8 or 10 or more different dsRNAs are produced in a cell or plant comprising the cell that have a pathogen-inhibitory effect. The dsRNAs may be expressed from multiple constructs introduced in different transformation events or could be introduced on a single nucleic acid molecule. The dsRNAs may be expressed using a single promoter or multiple different promoters. In one embodiment of the invention, single dsRNAs are produced that comprise nucleic acids homologous to multiple loci within a pathogen.

In still yet another aspect, the invention provides a recombinant host cell having in its genome at least one recombinant DNA sequence that is transcribed to produce at least one dsRNA molecule that functions when taken up by a plant pathogen to inhibit the expression of a target gene in the pathogen. The dsRNA molecule may be encoded by any of the nucleic acids described herein and as set forth in the sequence listing. The present invention also provides a transformed plant cell having in its genome at least one recombinant DNA sequence described herein. Transgenic plants comprising such a transformed plant cell are also provided, including progeny plants of any generation, seeds, and plant products, each comprising the recombinant DNA. The dsRNA molecules of the present invention may be found in the transgenic plant cell, for instance in the cytoplasm. They may also be found in an apoplastic space.

The methods and compositions of the present invention may be applied to any monocot and dicot plant, depending on the pathogen control desired. Specifically, the plants are intended to include, without limitation, alfalfa, aneth, apple, apricot, artichoke, arugula, asparagus, avocado, banana, barley, beans, beet, blackberry, blueberry, broccoli, brussel sprouts, cabbage, canola, cantaloupe, carrot, cassava, cauliflower, celery, cherry, chestnut, chickpea, cilantro, citrus, clementine, coffee, corn, cotton, cowpea, cucumber, Douglas fir, eggplant, endive, escarole, eucalyptus, fennel, figs, gourd, grape, grapefruit, honey dew, jicama, kiwifruit, lettuce, leeks, lemon, lime, Loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rhododendron, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini plants.

The invention also provides combinations of methods and compositions for controlling infection by plant pathogenic microorganisms. One means provides a dsRNA method as described herein for protecting plants from pathogen infection along with one or more chemical agents that exhibit features different from those exhibited by the dsRNA methods and compositions.

The present invention therefore provides a composition that contains two or more different agents each toxic to the same plant pathogenic microorganism, at least one of which comprises a dsRNA described herein. In certain embodiments, the second agent can be an agent selected from the group consisting of inhibitors of metabolic enzymes involved in amino acid or carbohydrate synthesis; inhibitors of cell division; cell wall synthesis inhibitors; inhibitors of DNA or RNA synthesis, gyrase inhibitors, tubulin assembly inhibitors, inhibitors of ATP synthesis; oxidative phosphorylation uncouplers; inhibitors of protein synthesis; MAP kinase inhibitors; lipid synthesis or oxidation inhibitors; sterol synthesis inhibitors; and melanin synthesis inhibitors.

A ribonucleic acid that is provided in a food source can be provided in an artificial medium formulated to meet particular nutritional requirements for maintaining an organism on such media. The medium may be supplemented with a pathogen controlling amount of an RNA that has been purified from a separate expression system to determine a pathogen controlling amount of RNA composition or to determine extent of suppressive activity when the supplemented diet is taken up. The diet can also be a recombinant cell transformed with a DNA sequence constructed for expression of the agent, the RNA, or the gene suppression agent. When the contents of one or more such transformed cells is taken up by the pathogen, a desired genotypic or phenotypic result is observed, indicating that the agent has functioned to inhibit the expression of a target nucleotide sequence that is within the cells of the pathogen.

A gene targeted for suppression can encode an essential protein, the predicted function of which is selected from the group consisting of ion regulation and transport, enzyme synthesis, maintenance of cell membrane potential, amino acid biosynthesis, amino acid degradation, development and differentiation, infection, penetration, development of appressoria or haustoria, mycelial growth, melanin synthesis, toxin synthesis, siderophore synthesis, sporulation, fruiting body synthesis, cell division, energy metabolism, respiration, and apoptosis, among others.

The invention further provides agronomically and commercially important products and/or compositions of matter including, but not limited to, animal feed, commodities, products and by-products that are intended for use as food for human consumption or for use in compositions and commodities that are intended for human consumption including but not limited to grain, flour, meal, starch, silage, extracted sugars and syrup, seed oil, cereals, and the like. The compositions and methods of making such products are well known in the art. In specific embodiments, a food or feed composition of the invention may be defined as obtained from a plant selected from soybean, rice, wheat, oat, barley, cotton, canola, chickpea, cowpea, and potato as applicable.

Such compositions may be defined as containing detectable amounts of a nucleotide sequence set forth herein, and thus are also diagnostic for any transgenic event containing such nucleotide sequences. These products are more likely to be derived from crops propagated with fewer pesticides and organophosphates as a result of their incorporation of the nucleotides of the present invention for controlling plant disease. Such commodities and commodity products can be produced from seed produced from a transgenic plant, wherein the transgenic plant expresses RNA from one or more contiguous nucleotides of the present invention or nucleotides of one or more plant pathogens, and the complements thereof. Such commodities and commodity products may also be useful in controlling pathogens of such commodity and commodity products, because of the presence in the commodity or commodity product of the pathogen gene suppressive RNA expressed from a gene sequence as set forth in the present invention.

The invention also provides a computer readable medium having recorded thereon one or more of the nucleotide sequences as set forth in SEQ ID NO:1-35, or complements thereof, for use in a number of computer based applications, including but not limited to DNA identity and similarity searching, protein identity and similarity searching, transcription profiling characterizations, comparisons between genomes, and artificial hybridization analyses.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIG. 1: Schematic representation of construct designed to stabilize dsRNA molecules with GC rich clamp region at each end of molecule.

FIG. 2: Schematic of design for stabilizing hairpin molecules.

FIG. 3: Alternative design for stabilizing hairpin molecules.

FIG. 4: Schematic of alternative design for stabilizing a dsRNA with two regions of a given gene, or two independent genes separated by a spacer region with clamp (C1 or C2) on either end.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention.

The present invention provides methods and compositions for genetic control of plant pathogen infections. DNA plasmid vectors encoding dsRNA molecules are designed to suppress fungal genes essential for growth, pathogenicity, and/or virulence. For example, the present invention provides recombinant DNA technologies to post-transcriptionally repress or inhibit expression of a target coding sequence in a pathogen to provide a pathogen-protective effect by having the pathogen take up one or more double stranded or small interfering ribonucleic acid (RNA) molecules transcribed from all or a portion of a target coding sequence, thereby controlling the infection. Therefore, the present invention relates to sequence-specific inhibition of expression of coding sequences using double-stranded RNA (dsRNA), including small interfering RNA (siRNA), to achieve the intended levels of pathogen control.

Isolated and substantially purified nucleic acid molecules including but not limited to non-naturally occurring nucleotide sequences and recombinant DNA constructs for transcribing dsRNA molecules of the present invention are provided that suppress or inhibit the expression of an endogenous coding sequence or a target coding sequence in the pathogen when (1) introduced thereto, (2) provided in the environment of said pathogen, or (3) when said pathogen is contacted by said dsRNA. Transgenic plants that (a) contain nucleotide sequences encoding the isolated and substantially purified nucleic acid molecules and the non-naturally occurring recombinant DNA constructs for transcribing the dsRNA molecules for controlling plant pathogen infections, and (b) display resistance and/or enhanced tolerance to the infections, are also provided. Compositions containing the dsRNA nucleotide sequences of the present invention for use in topical applications onto plants or onto animals or into the environment of an animal to achieve the elimination or reduction of plant pathogen infection are also described.

cDNA sequences encoding proteins or parts of proteins essential for survival, such as amino acid sequences involved in various metabolic or catabolic biochemical pathways, cell division, reproduction, energy metabolism, digestion, and the like may be selected for use in preparing double stranded RNA molecules to be provided in the host plant of a pathogenic microorganism. As described herein, taking up of compositions by a target organism containing one or more dsRNAs, at least one segment of which corresponds to at least a substantially identical segment of RNA produced in the cells of the target pathogen, resulted in death, or other inhibition of the target. These results indicated that a nucleotide sequence, either DNA or RNA, derived from a plant pathogen can be used to construct plant cells resistant to infestation by the pathogen. The host plant of the pathogen, for example, can be transformed to contain one or more of the nucleotide sequences derived from the pathogen. The nucleotide sequence transformed into the host may encode one or more RNAs that form into a dsRNA sequence in the cells or biological fluids within the transformed host, thus making the dsRNA available if/when the pathogen forms a nutritional relationship with the transgenic host, resulting in the suppression of expression of one or more genes in the cells of the pathogen and ultimately the death or inhibition of growth of the pathogen

The present invention relates generally to genetic control of plant pathogens in host organisms. More particularly, the present invention includes the methods for delivery of pathogen control agents to a plant pathogenic microorganism. Such control agents cause, directly or indirectly, an impairment in the ability of the pathogen to maintain itself, grow or otherwise cause disease in a target host. The present invention provides methods for employing stabilized dsRNA molecules to the pathogen as a means for suppression of targeted genes in the pathogen, thus achieving desired control of plant disease in the host targeted by the pathogen.

In accomplishing the foregoing, the present invention provides a method of inhibiting expression of a target gene in a plant pathogenic microorganism, including for example, rust fungi, resulting in the cessation of infection, growth, development, reproduction, infectivity, and eventually may result in the death of the pathogen. The method comprises in one embodiment introducing partial or fully stabilized double-stranded RNA (dsRNA) nucleotide molecules into a nutritional composition that the pathogen relics on as a food source, and making the nutritional composition available to the pathogen for feeding. Taking up a nutritional composition containing the double stranded or siRNA molecules results in the inhibition of expression of at least one target gene in the cells of the pathogen. Inhibition of the target gene exerts a deleterious effect upon the pathogen.

In certain embodiments, dsRNA molecules provided by the invention comprise nucleotide sequences complementary to a sequence as set forth in any of SEQ ID NO:3-15; SEQ ID NO:18-23; SEQ ID NO:29, and SEQ ID NO:33-35, or fragments thereof, the inhibition of which in a pathogen organism results in the reduction or removal of a protein or nucleotide sequence agent that is essential for the pathogen's growth and development or other biological function. The nucleotide sequence selected may exhibit from about 80% to at least about 100% sequence identity to one of the nucleotide sequences as set forth in SEQ ID NO:3-15; SEQ ID NO:18-23; SEQ ID NO:29, and SEQ ID NO:33-35, or fragments thereof. Such inhibition can be described as specific in that a nucleotide sequence from a portion of the target gene is chosen from which the inhibitory dsRNA or siRNA is transcribed. The method is effective in inhibiting the expression of at least one target gene and can be used to inhibit many different types of target genes in the pathogen.

The sequences identified as having a pathogen protective effect may be readily expressed as dsRNA molecules through the creation of appropriate expression constructs. For example, such sequences can be expressed as a hairpin and stem and loop structure by taking a first segment corresponding to a sequence selected from SEQ ID NO:3-15; SEQ ID NO:18-23; SEQ ID NO:29, and SEQ ID NO:33-35 or a fragment thereof, linking this sequence to a second segment spacer region that is not homologous or complementary to the first segment, and linking this to a third segment that transcribes an RNA, wherein at least a portion of the third segment is substantially complementarity to the first segment. Such a construct forms a stem and loop structure by hybridization of the first segment with the third segment and a loop structure forms comprising the second segment (WO94/01550, WO98/05770, US 2002/0048814A1, and US 2003/0018993A1).

A. Nucleic Acid Compositions and Constructs

The invention provides recombinant DNA constructs for use in achieving stable transformation of particular host targets. Transformed host targets may express effective levels of preferred dsRNA or siRNA molecules from the recombinant DNA constructs. Pairs of isolated and purified nucleotide sequences may be provided from cDNA library and/or genomic library information. The pairs of nucleotide sequences may be derived from any preferred invertebrate pathogen for use as thermal amplification primers to generate the dsRNA and siRNA molecules of the present invention.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides or peptides.

Provided according to the invention are nucleotide sequences, the expression of which results in an RNA sequence which is substantially homologous to an RNA molecule of a targeted gene in a pathogen that comprises an RNA sequence encoded by a nucleotide sequence within the genome of the insect. Thus, after taking up the stabilized RNA sequence, down-regulation of the nucleotide sequence of the target gene in the cells of the pathogen may be obtained resulting in a deleterious effect on the maintenance, viability, proliferation, or reproduction of the pathogen.

As used herein, the term “substantially homologous” or “substantial homology”, with reference to a nucleic acid sequence, includes a nucleotide sequence that hybridizes under stringent conditions to the coding sequence as set forth in any of SEQ ID NO:3-15; SEQ ID NO:18-23; and SEQ ID NO:29 as set forth in the sequence listing, or the complements thereof. Sequences that hybridize under stringent conditions to any of SEQ ID NO:3-15; SEQ ID NO:18-23; SEQ ID NO:29, and SEQ ID NO:33-35 as set forth in the sequence listing, or the complements thereof, are those that allow an antiparallel alignment to take place between the two sequences, and the two sequences are then able, under stringent conditions, to form hydrogen bonds with corresponding bases on the opposite strand to form a duplex molecule that is sufficiently stable under the stringent conditions to be detectable using methods well known in the art. Substantially homologous sequences have preferably from about 70% to about 80% sequence identity, or more preferably from about 80% to about 85% sequence identity, or most preferable from about 90% to about 95% sequence identity, to about 99% sequence identity, to the referent nucleotide sequences as set forth in any of SEQ ID NO:3-15; SEQ ID NO:18-23; SEQ ID NO:29, and SEQ ID NO:33-35 as set forth in the sequence listing, or the complements thereof.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, a “comparison window” refers to a conceptual segment of at least 6 contiguous positions, usually about 50 to about 100, more usually about 100 to about 150, in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences Those skilled in the art should refer, for example, to the detailed methods used for sequence alignment in the Wisconsin Genetics Software Package Release 7.0, Genetics Computer Group, 575 Science Drive Madison, Wis., USA).

The present invention provides DNA sequences capable of being expressed as an RNA in a cell or microorganism to inhibit target gene expression in a cell, tissue or organ of a pathogenic microorganism. The sequences comprises a DNA molecule coding for one or more different nucleotide sequences, wherein each of the different nucleotide sequences comprises a sense nucleotide sequence and an antisense nucleotide sequence connected by a spacer sequence coding for a dsRNA molecule of the present invention. The spacer sequence constitutes part of the sense nucleotide sequence or the antisense nucleotide sequence and forms within the dsRNA molecule between the sense and antisense sequences. The sense nucleotide sequence or the antisense nucleotide sequence is substantially identical to the nucleotide sequence of the target gene or a derivative thereof or a complementary sequence thereto. The dsDNA molecule may be placed operably under the control of a promoter sequence that functions in the cell, tissue or organ of the host expressing the dsDNA to produce dsRNA molecules. In one embodiment, the DNA sequence may be derived from a nucleotide sequence as set forth in SEQ ID NO:3-15; SEQ ID NO:18-23; SEQ ID NO:29; and SEQ ID NO:33-35 in the sequence listing.

The invention also provides a DNA sequence for expression in a cell of a plant that, upon expression of the DNA to RNA and being taken up by a pathogen achieves suppression of a target gene in a cell, tissue or organ of a pathogen. The dsRNA at least comprises one or multiple structural gene sequences, wherein each of the structural gene sequences comprises a sense nucleotide sequence and an antisense nucleotide sequence connected by a spacer sequence that forms a loop within the complementary and antisense sequences. The sense nucleotide sequence or the antisense nucleotide sequence is substantially identical to the nucleotide sequence of the target gene, derivative thereof, or sequence complementary thereto. The one or more structural gene sequences is placed operably under the control of one or more promoter sequences, at least one of which is operable in the cell, tissue or organ of a prokaryotic or eukaryotic organism, particularly a plant pathogenic fungus.

A gene sequence or fragment for pathogen control according to the invention may be cloned between two tissue specific promoters, such as two root specific promoters which are operable in a transgenic plant cell and therein expressed to produce mRNA in the transgenic plant cell that form dsRNA molecules thereto. The dsRNA molecules contained in plant tissues are taken up by a pathogen so that the intended suppression of the target gene expression is achieved.

A nucleotide sequence provided by the present invention may comprise an inverted repeat separated by a “spacer sequence.” The spacer sequence may be a region comprising any sequence of nucleotides that facilitates secondary structure formation between each repeat, where this is required. In one embodiment of the present invention, the spacer sequence is part of the sense or antisense coding sequence for mRNA. The spacer sequence may alternatively comprise any combination of nucleotides or homologues thereof that are capable of being linked covalently to a nucleic acid molecule. The spacer sequence may comprise a sequence of nucleotides of at least about 10-100 nucleotides in length, or alternatively at least about 100-200 nucleotides in length, at least 200-400 about nucleotides in length, or at least about 400-500 nucleotides in length.

The nucleic acid molecules or fragment of the nucleic acid molecules or other nucleic acid molecules in the sequence listing are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are described by Sambrook, et al., (1989), and by Haymes et al., (1985).

Departures from complete complementarity are therefore permissible, as long as such departures do not completely preclude the capacity of the molecules to form a double-stranded structure. Thus, in order for a nucleic acid molecule or a fragment of the nucleic acid molecule to serve as a primer or probe it needs only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Such conditions thus include, for example, 0.2×SSC at 65° C.

Both temperature and salt may be varied, or either the temperature or the salt concentration may be held constant while the other variable is changed. A nucleic acid for use in the present invention may specifically hybridize to one or more of nucleic acid molecules from WCR or complements thereof under such conditions. Preferably, a nucleic acid for use in the present invention will exhibit at least from about 80%, or at least from about 90%, or at least from about 95%, or at least from about 98% or even about 100% sequence identity with one or more nucleic acid molecules as set forth in SEQ ID NO:3-15; SEQ ID NO:18-23; SEQ ID NO:29; and SEQ ID NO:33-35 as set forth in the sequence listing.

Nucleic acids of the present invention may also be synthesized, either completely or in part, especially where it is desirable to provide plant-preferred sequences, by methods known in the art. Thus, all or a portion of the nucleic acids of the present invention may be synthesized using codons preferred by a selected host. Species-preferred codons may be determined, for example, from the codons used most frequently in the proteins expressed in a particular host species. Other modifications of the nucleotide sequences may result in mutants having slightly altered activity.

dsRNA or siRNA nucleotide sequences comprise double strands of polymerized ribonucleotide and may include modifications to either the phosphate-sugar backbone or the nucleoside. Modifications in RNA structure may be tailored to allow specific genetic inhibition. In one embodiment, the dsRNA molecules may be modified through an enzymatic process so that siRNA molecules may be generated. The siRNA can efficiently mediate the down-regulation effect for some target genes in some pathogens. This enzymatic process may be accomplished by utilizing an RNAse III enzyme or a DICER enzyme, present in the cells of an insect, a vertebrate animal, a fungus or a plant in the eukaryotic RNAi pathway (Elbashir et al., 2002; Hamilton and Baulcombe, 1999). This process may also utilize a recombinant DICER or RNAse III introduced into the cells of a target insect through recombinant DNA techniques that are readily known to those skilled in the art. Both the DICER enzyme and RNAse III, being naturally occurring in a pathogen or being made through recombinant DNA techniques, cleave larger dsRNA strands into smaller oligonucleotides. The DICER enzymes specifically cut the dsRNA molecules into siRNA pieces each of which is about 19-25 nucleotides in length while the RNAse III enzymes normally cleave the dsRNA molecules into 12-15 base-pair siRNA. The siRNA molecules produced by the either of the enzymes have 2 to 3 nucleotide 3′ overhangs, and 5′ phosphate and 3′ hydroxyl termini. The siRNA molecules generated by RNAse III enzyme are substantially the same as those produced by Dicer enzymes in the eukaryotic RNAi pathway and are hence then targeted and degraded by an inherent cellular RNA-degrading mechanism after they are subsequently unwound, separated into single-stranded RNA and hybridized with the RNA sequences transcribed by the target gene. This process results in the effective degradation or removal of the RNA sequence encoded by the nucleotide sequence of the target gene in the pathogen. The outcome is the silencing of a particularly targeted nucleotide sequence within the pathogen. Detailed descriptions of enzymatic processes can be found in Hannon (2002).

A nucleotide sequence of the present invention can be recorded on computer readable media. As used herein, “computer readable media” refers to any tangible medium of expression that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; optical character recognition formatted computer files, and hybrids of these categories such as magnetic/optical storage media. A skilled artisan can readily appreciate that any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII text file, stored in a database application, such as DB2, Sybase, Oracle, or the like. The skilled artisan can readily adapt any number of data processor structuring formats (e.g. text file or database) in order to obtain computer readable medium having recorded thereon the nucleotide sequence information of the present invention.

Computer software is publicly available which allows a skilled artisan to access sequence information provided in a computer readable medium. Software that implements the BLAST (Altschul et al., 1990) and BLAZE (Brutlag, et al., 1993) search algorithms on a Sybase system can be used to identify open reading frames (ORFs) within sequences such as the Unigenes and EST's that are provided herein and that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As used herein, “a target structural motif,” or “target motif,” refers to any rationally selected sequence or combination of sequences in which the sequence or sequences are chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

B. Recombinant Vectors and Host Cell Transformation

A recombinant DNA vector may, for example, be a linear or a closed circular plasmid. The vector system may be a single vector or plasmid or two or more vectors or plasmids that together contain the total DNA to be introduced into the genome of the host. In addition, a vector may be an expression vector. Nucleic acid molecules as set forth in SEQ ID NO:3-15; SEQ ID NO:18-23; SEQ ID NO:29; and SEQ ID NO:33-35 or fragments thereof can, for example, be suitably inserted into a vector under the control of a suitable promoter that functions in one or more hosts to drive expression of a linked coding sequence or other DNA sequence. Many vectors are available for this purpose, and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more selectable marker genes, and an inducible promoter allowing the expression of exogenous DNA.

Expression and cloning vectors generally contain a selection gene, also referred to as a selectable marker. This gene encodes a protein necessary for the survival or growth of transformed host cells grown in a selective culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. Those cells that are successfully transformed with a heterologous protein or fragment thereof produce a protein conferring drug resistance and thus survive the selection regimen.

An expression vector for producing a mRNA or a dsRNA can also contain an inducible promoter that is recognized by the host bacterial organism and is operably linked to the nucleic acid encoding, for example, the nucleic acid molecule coding a fungal (e.g. S. sclerotiorum) mRNA or fragment thereof of interest. Inducible promoters suitable for use with bacterial hosts include β-lactamase promoter, E. coli λ phage PL and PR promoters, and E. coli galactose promoter, arabinose promoter, alkaline phosphatase promoter, tryptophan (trp) promoter, and the lactose operon promoter and variations thereof and hybrid promoters such as the tac promoter. However, other known inducible bacterial promoters are suitable.

The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream (5′ noncoding sequences), within, or downstream (3′ non-translated sequences) of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, and polyadenylation recognition sequences and the like.

Alternatively, the expression constructs can be integrated into the bacterial genome with an integrating vector. Integrating vectors typically contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP 0 127,328). Integrating vectors may also be comprised of bacteriophage or transposon sequences. Suicide vectors are also known in the art.

Construction of suitable vectors containing one or more of the above-listed components employs standard recombinant DNA techniques. Isolated plasmids or DNA fragments are cleaved, tailored, and re-ligated in the form desired to generate the plasmids required. Examples of available bacterial expression vectors include, but are not limited to, the multifunctional E. coli cloning and expression vectors such as Bluescript™ (Stratagene, La Jolla, Calif.), in which, for example, a nucleic acid encoding for a S. sclerotiorum protein or fragment thereof, may be ligated into the vector in frame with sequences for the amino-terminal Met and the subsequent 7 residues of β-galactosidase so that a hybrid protein is produced; pIN vectors (Van Heeke and Schuster, 1989); and the like.

A yeast recombinant construct can typically include one or more of the following: a promoter sequence, fusion partner sequence, leader sequence, transcription termination sequence, a selectable marker. These elements can be combined into an expression cassette, which may be maintained in a replicon, such as an extrachromosomal element (e.g., plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 (Botstein et al., 1979), pCl/1 (Brake et al., 1984), and YRp17 (Stinchcomb et al., 1982). In addition, a replicon may be either a high or low copy number plasmid. A high copy number plasmid will generally have a copy number ranging from about 5 to about 200, and typically about 10 to about 150. A host containing a high copy number plasmid will preferably have at least about 10, and more preferably at least about 20 copies of the plasmid.

Useful yeast promoter sequences can be derived from genes encoding enzymes in the metabolic pathway. Examples of such genes include alcohol dehydrogenase (ADH) (EP 0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EP 329 203). The yeast PHOS gene, encoding acid phosphatase, also provides useful promoter sequences (Myanohara et al., 1983). In addition, synthetic promoters that do not occur in nature also function as yeast promoters. Examples of such hybrid promoters include the ADH regulatory sequence linked to the GAP transcription activation region (U.S. Pat. Nos. 4,876,197 and 4,880,734). Examples of transcription terminator sequences and other yeast-recognized termination sequences, such as those coding for glycolytic enzymes, are known to those of skill in the art.

Alternatively, the expression constructs can be integrated into the yeast genome with an integrating vector. Integrating vectors typically contain at least one sequence homologous to a yeast chromosome that allows the vector to integrate, and preferably contain two homologous sequences flanking the expression construct. Integrations appear to result from recombinations between homologous DNA in the vector and the yeast chromosome (Orr-Weaver et al., 1983). An integrating vector may be directed to a specific locus in yeast by selecting the appropriate homologous sequence for inclusion in the vector. See On-Weaver et al., supra. One or more expression constructs may integrate, possibly affecting levels of recombinant protein produced (Rine et al., 1983).

The present invention also contemplates transformation of a nucleotide sequence of the present invention into a plant to achieve pathogen inhibitory levels of expression of one or more dsRNA molecules. A transformation vector can be readily prepared using methods available in the art. The transformation vector comprises one or more nucleotide sequences that is/are capable of being transcribed to an RNA molecule and that is/are substantially homologous and/or complementary to one or more nucleotide sequences encoded by the genome of the pathogen, such that upon uptake of the RNA transcribed from the one or more nucleotide sequences by the pathogen, there is down-regulation of expression of at least one of the respective nucleotide sequences of the genome of the pathogen.

The transformation vector may be termed a dsDNA construct and may also be defined as a recombinant molecule, a disease control agent, a genetic molecule or a chimeric genetic construct. A chimeric genetic construct of the present invention may comprise, for example, nucleotide sequences encoding one or more antisense transcripts, one or more sense transcripts, one or more of each of the aforementioned, wherein all or part of a transcript therefrom is homologous to all or part of an RNA molecule comprising an RNA sequence encoded by a nucleotide sequence within the genome of a pathogen.

In one embodiment the plant transformation vector comprises an isolated and purified DNA molecule comprising a heterologous promoter operatively linked to one or more nucleotide sequences of the present invention. The nucleotide sequence is selected from the group consisting of SEQ ID NO:3-15; SEQ ID NO:18-23; SEQ ID NO:29; and SEQ ID NO:33-35, as set forth in the sequence listing, or a fragment thereof. The nucleotide sequence includes a segment coding all or part of an RNA present within a targeted pathogen RNA transcript and may comprise inverted repeats of all or a part of a targeted pathogen RNA. The DNA molecule comprising the expression vector may also contain a functional intron sequence positioned either upstream of the coding sequence or even within the coding sequence, and may also contain a five prime (5′) untranslated leader sequence (i.e., a UTR or 5′-UTR) positioned between the promoter and the point of translation initiation.

A plant transformation vector may contain sequences from more than one gene, thus allowing production of more than one dsRNA for inhibiting expression of two or more genes in cells of a target pathogen. One skilled in the art will readily appreciate that segments of DNA whose sequence corresponds to that present in different genes can be combined into a single composite DNA segment for expression in a transgenic plant. Alternatively, a plasmid of the present invention already containing at least one DNA segment can be modified by the sequential insertion of additional DNA segments between the enhancer and promoter and terminator sequences. In the disease control agent of the present invention designed for the inhibition of multiple genes, the genes to be inhibited can be obtained from the same pathogen species in order to enhance the effectiveness of the control agent. In certain embodiments, the genes can be derived from different pathogens in order to broaden the range of pathogens against which the agent(s) is/are effective. When multiple genes are targeted for suppression or a combination of expression and suppression, a polycistronic DNA element can be fabricated as illustrated and disclosed in Fillatti, Application Publication No. US 2004-0029283.

Promoters that function in different plant species are also well known in the art. Promoters useful for expression of polypeptides in plants include those that are inducible, viral, synthetic, or constitutive as described in Odell et al. (1985), and/or promoters that are temporally regulated, spatially regulated, and spatio-temporally regulated. Preferred promoters include the enhanced CaMV35S promoters, and the FMV35S promoter. For the purpose of the present invention, it may be preferable to achieve the highest levels of expression of these genes within the leaves or photosynthetic tissues of plants. A number of leaf-specific promoters have been identified and are known in the art (e.g. Stahl et al. 2004; Busk 1997).

A recombinant DNA vector or construct of the present invention will typically comprise a selectable marker that confers a selectable phenotype on plant cells. Selectable is markers may also be used to select for plants or plant cells that contain the exogenous nucleic acids encoding polypeptides or proteins of the present invention. The marker may encode biocide resistance, antibiotic resistance (e.g., kanamycin, G418 bleomycin, hygromycin, etc.), or herbicide resistance (e.g., glyphosate, etc.). Examples of selectable markers include, but are not limited to, a neo gene which codes for kanamycin resistance and can be selected for using kanamycin, G418, etc., a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulfonylurea resistance; and a methotrexate resistant DHFR gene. Examples of such selectable markers are illustrated in U.S. Pat. Nos. 5,550,318; 5,633,435; 5,780,708 and 6,118,047.

A recombinant vector or construct of the present invention may also include a screenable marker. Screenable markers may be used to monitor expression. Exemplary screenable markers include a β-glucuronidase or uidA gene (GUS) which encodes an enzyme for which various chromogenic substrates are known (Jefferson, 1987; Jefferson et al., 1987); an R-locus gene, which encodes a product that regulates the production of anthocyanin pigments (red color) in plant tissues (Dellaporta et al., 1988); a β-lactamase gene (Sutcliffe et al., 1978), a gene which encodes an enzyme for which various chromogenic substrates are known (e.g., PADAC, a chromogenic cephalosporin); a luciferase gene (Ow et al., 1986) a xylE gene (Zukowsky et al., 1983) which encodes a catechol dioxygenase that can convert chromogenic catechols; an α-amylase gene (Ikatu et al., 1990); a tyrosinase gene (Katz et al., 1983) which encodes an enzyme capable of oxidizing tyrosine to DOPA and dopaquinone which in turn condenses to melanin; an α-galactosidase, which catalyzes a chromogenic galactose substrate.

Preferred plant transformation vectors include those derived from a Ti plasmid of Agrobacterium tumefaciens (e.g. U.S. Pat. Nos. 4,536,475, 4,693,977, 4,886,937, 5,501,967 and EP 0 122 791). Agrobacterium rhizogenes plasmids (or “Ri”) are also useful and known in the art. Other preferred plant transformation vectors include those disclosed, e.g., by Herrera-Estrella (1983); Bevan (1983), Klee (1985) and EP 0 120 516.

In general it is preferred to introduce a functional recombinant DNA at a non-specific location in a plant genome. In special cases it may be useful to insert a recombinant DNA construct by site-specific integration. Several site-specific recombination systems exist which are known to function implants include cre-lox as disclosed in U.S. Pat. No. 4,959,317 and FLP-FRT as disclosed in U.S. Pat. No. 5,527,695.

Suitable methods for transformation of host cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253), by agitation with silicon carbide fibers (Kaeppler et al., 1990; U.S. Pat. No. 5,302,523; and U.S. Pat. No. 5,464,765), by Agrobacterium-mediated transformation (U.S. Pat. No. 5,591,616 and U.S. Pat. No. 5,563,055) and by acceleration of DNA coated particles (U.S. Pat. No. 5,550,318; U.S. Pat. No. 5,538,877; and U.S. Pat. No. 5,538,880), etc. Through the application of techniques such as these, the cells of virtually any species may be stably transformed. In the case of multicellular species, the transgenic cells may be regenerated into transgenic organisms.

Methods for the creation of transgenic plants and expression of heterologous nucleic acids in plants in particular are known and may be used with the nucleic acids provided herein to prepare transgenic plants that exhibit reduced susceptibility to feeding by a target pathogen organism such as a rust fungus. Plant transformation vectors can be prepared, for example, by inserting the dsRNA producing nucleic acids disclosed herein into plant transformation vectors and introducing these into plants. One known vector system has been derived by modifying the natural gene transfer system of Agrobacterium tumefaciens. The natural system comprises large Ti (tumor-inducing)-plasmids containing a large segment, known as T-DNA, which is transferred to transformed plants. Another segment of the Ti plasmid, the vir region, is responsible for T-DNA transfer. The T-DNA region is bordered by terminal repeats. In the modified binary vectors the tumor-inducing genes have been deleted and the functions of the vir region are utilized to transfer foreign DNA bordered by the T-DNA border sequences. The T-region may also contain a selectable marker for efficient recovery of transgenic plants and cells, and a multiple cloning site for inserting sequences for transfer such as a dsRNA encoding nucleic acid.

A transgenic plant formed using Agrobacterium transformation methods typically contains a single simple recombinant DNA sequence inserted into one chromosome and is referred to as a transgenic event. Such transgenic plants can be referred to as being heterozygous for the inserted exogenous sequence. A transgenic plant homozygous with respect to a transgene can be obtained by sexually mating (selfing) an independent segregant transgenic plant that contains a single exogenous gene sequence to itself, for example an F0 plant, to produce F1 seed. One fourth of the F1 seed produced will be homozygous with respect to the transgene. Germinating F1 seed results in plants that can be tested for heterozygosity, typically using a SNP assay or a thermal amplification assay that allows for the distinction between heterozygotes and homozygotes (i.e., a zygosity assay). Crossing a heterozygous plant with itself or another heterozygous plant results in only heterozygous progeny.

C. Nucleic Acid Expression and Target Gene Suppression

The present invention provides, as an example, a transformed host plant of a pathogenic target organism, transformed plant cells and transformed plants and their progeny. The transformed plant cells and transformed plants may be engineered to express one or more of the dsRNA or siRNA sequences, under the control of a heterologous promoter, described herein to provide a pathogen-protective effect. These sequences may be used for gene suppression in a pathogen, thereby reducing the level or incidence of disease caused by the pathogen on a protected transformed host organism. As used herein the words “gene suppression” are intended to refer to any of the well-known methods for reducing the levels of protein produced as a result of gene transcription to mRNA and subsequent translation of the mRNA. Thus the term “gene suppressive amount” refers to an amount of active agent sufficient to suppress the level of a given protein product and/or mRNA in a cell.

Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi).

Transcriptional suppression is mediated by the presence in the cell of a dsRNA gene suppression agent exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pathogens that may take up or contact plant material containing gene suppression agents, specifically designed to inhibit or suppress the expression of one or more homologous or complementary sequences in the cells of the pathogen. Post-transcriptional gene suppression by anti-sense or sense oriented RNA to regulate gene expression in plant cells is disclosed in U.S. Pat. Nos. 5,107,065, 5,759,829, 5,283,184, and 5,231,020. The use of dsRNA to suppress genes in plants is disclosed in WO 99/53050, WO 99/49029, U.S. Patent Application Publication No. 2003/0175965, and 2003/0061626, U.S. patent application Ser. No. 10/465,800, and U.S. Pat. Nos. 6,506,559, and 6,326,193.

A beneficial method of post transcriptional gene suppression in plants employs both sense-oriented and anti-sense-oriented, transcribed RNA which is stabilized, e.g., as a hairpin and stem and loop structure. A preferred DNA construct for effecting post transcriptional gene suppression is one in which a first segment encodes an RNA exhibiting an anti-sense orientation exhibiting substantial identity to a segment of a gene targeted for suppression, which is linked to a second segment encoding an RNA exhibiting substantial complementarity to the first segment. Such a construct forms a stem and loop structure by hybridization of the first segment with the second segment and a loop structure from the nucleotide sequences linking the two segments (see WO94/01550, WO98/05770, US 2002/0048814, and US 2003/0018993). Additional examples of constructs that express stabilized RNA are also found in Example 8.

According to one embodiment of the present invention, there is provided a nucleotide sequence, for which in vitro expression results in transcription of a stabilized RNA sequence that is substantially homologous to an RNA molecule of a targeted gene in a pathogen that comprises an RNA sequence encoded by a nucleotide sequence within the genome of the pathogen. Thus, after the pathogen takes up the stabilized RNA sequence, a down-regulation of expression of the nucleotide sequence corresponding to the target gene in the cells of a target pathogen is affected.

Inhibition of a target gene using the stabilized dsRNA technology of the present invention is sequence-specific in that nucleotide sequences corresponding to the duplex region of the RNA are targeted for genetic inhibition. RNA containing a nucleotide sequences identical to a portion of the target gene is preferred for inhibition. RNA sequences with insertions, deletions, and single point mutations relative to the target sequence have also been found to be effective for inhibition. In performance of the present invention, it is preferred that the inhibitory dsRNA and the portion of the target gene share at least from about 80% sequence identity, or from about 90% sequence identity, or from about 95% sequence identity, or from about 99% sequence identity, or even about 100% sequence identity. Alternatively, the duplex region of the RNA may be defined functionally as a nucleotide sequence that is capable of hybridizing with a portion of the target gene transcript. A less than full length sequence exhibiting a greater homology compensates for a longer less homologous sequence. The length of the identical nucleotide sequences may be at least about 25, 50, 100, 200, 300, 400, 500 or at least about 1000 bases. Normally, a sequence of greater than 20-100 nucleotides should be used, though a sequence of greater than about 200-300 nucleotides would be preferred, and a sequence of greater than about 500-1000 nucleotides would be especially preferred depending on the size of the target gene. The invention has the advantage of being able to tolerate sequence variations that might be expected due to genetic mutation, strain polymorphism, or evolutionary divergence. The introduced nucleic acid molecule may not need to be absolute homology, may not need to be full length, relative to either the primary transcription product or fully processed mRNA of the target gene.

Inhibition of target gene expression may be quantified by measuring either the endogenous target RNA or the protein produced by translation of the target RNA and the consequences of inhibition can be confirmed by examination of the outward properties of the cell or organism. Techniques for quantifying RNA and proteins are well known to one of ordinary skill in the art. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, and tetracyclin, and the like.

In certain embodiments gene expression is inhibited by at least 10%, preferably by at least 33%, more preferably by at least 50%, and yet more preferably by at least 80%. In particularly preferred embodiments of the invention gene expression is inhibited by at least 80%, more preferably by at least 90%, more preferably by at least 95%, or by at least 99% within cells in the pathogen so a significant inhibition takes place. Significant inhibition is intended to refer to sufficient inhibition that results in a detectable phenotype (e.g., cessation of vegetative or reproductive growth, mortality, etc.) or a detectable decrease in RNA and/or protein corresponding to the target gene being inhibited. Although in certain embodiments of the invention inhibition occurs in substantially all cells of the pathogen, in other preferred embodiments inhibition occurs in only a subset of cells expressing the gene. For example, if the target pathogen is a rust fungus and the gene to be inhibited plays an essential role in haustoria, inhibition of the gene within these cells is sufficient to exert a deleterious effect on the pathogen.

dsRNA molecules may be synthesized either in vivo or in vitro. The dsRNA may be formed by a single self-complementary RNA strand or from two complementary RNA strands. Endogenous RNA polymerase of the cell may mediate transcription in vivo, or cloned RNA polymerase can be used for transcription in vivo or in vitro Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

A RNA, dsRNA, siRNA, or miRNA of the present invention may be produced chemically or enzymatically by one skilled in the art through manual or automated reactions or in vivo in another organism. RNA may also be produced by partial or total organic synthesis; any modified ribonucleotide can be introduced by in vitro enzymatic or organic synthesis. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874, 5,698,425, 5,712,135, 5,789,214, and 5,804,693). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

For transcription from a transgene in vivo or an expression construct, a regulatory region (e.g., promoter, enhancer, silencer, and polyadenylation) may be used to transcribe the RNA strand (or strands). Therefore, in one embodiment, the nucleotide sequences for use in producing RNA molecules may be operably linked to one or more promoter sequences functional in a microorganism, a fungus or a plant host cell. Ideally, the nucleotide sequences are placed under the control of an endogenous promoter, normally resident in the host genome. The nucleotide sequence of the present invention, under the control of an operably linked promoter sequence, may further be flanked by additional sequences that advantageously affect its transcription and/or the stability of a resulting transcript. Such sequences are generally located upstream of the operably linked promoter and/or downstream of the 3′ end of the expression construct and may occur both upstream of the promoter and downstream of the 3′ end of the expression construct, although such an upstream sequence only is also contemplated.

As used herein, the term “disease control agent”, or “gene suppression agent” refers to a particular RNA molecule consisting at least of a first RNA segment and a second RNA segment, which may optionally be linked by a third RNA segment. The first and the second RNA segments can be independently expressed in the same cell from separate expression cassettes, forming dsRNA disease control or gene suppression agents upon hybridization to each other. When linked together by the third RNA segment, the first and the second RNA segments lie within the length of a single RNA molecule and are substantially inverted repeats of each other. The complementarity between the first and the second RNA segments results in the ability of the two segments to hybridize in vivo and in vitro to form a double stranded molecule, i.e., a stem, linked together at one end of each of the first and second segments by the third segment which forms a loop, so that the entire structure forms into a stem and loop structure, or even more tightly hybridizing structures may form into a stem-loop knotted structure. The first and the second segments correspond invariably and not respectively to a sense and an antisense sequence with respect to the target RNA transcribed from the target gene in the target pathogen. The control agent can also be a substantially purified (or isolated) nucleic acid molecule and more specifically nucleic acid molecules or nucleic acid fragment molecules thereof from a genomic DNA (gDNA) or cDNA library. Alternatively, the fragments may comprise smaller oligonucleotides having from about 15 to about 250 nucleotide residues, and more preferably, from about 15 to about 30 nucleotide residues.

As used herein, the term “genome” as it applies to cells of a pathogen or a host encompasses not only chromosomal DNA found within the nucleus, but organelle DNA found within subcellular components of the cell. The DNA's of the present invention introduced into plant cells can therefore be either chromosomally integrated or organelle-localized. The term “genome” as it applies to bacteria encompasses both the chromosome and plasmids within a bacterial host cell. The DNA's of the present invention introduced into bacterial host cells can therefore be either chromosomally integrated or plasmid-localized.

As used herein, the term “pathogen” refers to Ascomycetes, Basidiomycetes, Deuteromycetes, Oomycetes, and the like that are present in the environment and that may infect or cause disease on or in host plant material transformed to express or coated with a double stranded gene suppression agent containing the gene suppression agent. As used herein, “phytopathogenic microorganism” refers to microorganisms that can cause plant disease, including viruses, bacteria, fungi, oomycetes, chytrids, algae, and nematodes. As used herein, a “pathogen resistance” trait is a characteristic of a plant that causes the plant host to be resistant to attack from a pathogen that typically is capable of inflicting damage or loss to the plant. Such pathogen resistance can arise from a natural mutation or more typically from incorporation of recombinant DNA that confers pathogen resistance. To impart pathogen resistance to a transgenic plant a recombinant DNA can, for example, be transcribed into a RNA molecule that forms a dsRNA molecule within the tissues or fluids of the recombinant plant. The dsRNA molecule is comprised in part of a segment of RNA that is identical to a corresponding RNA segment encoded from a DNA sequence within a pathogen that prefers to cause disease on the recombinant plant. Expression of the gene within the target pathogen is suppressed by the dsRNA, and the suppression of expression of the gene in the target pathogen results in the plant being resistant to the pathogen. Fire et al., (U.S. Pat. No. 6,506,599) generically described inhibition of pest infestation, providing specifics only about several nucleotide sequences that were effective for inhibition of gene function in the nematode species Caenorhabditis elegans. Similarly, Plaetinck et al., (US 2003/0061626) describe the use of dsRNA for inhibiting gene function in a variety of nematode pests. Mesa et al., (US 2003/0150017) describe using dsDNA sequences to transform host cells to express corresponding dsRNA sequences that are substantially identical to target sequences in specific pests, and particularly describe constructing recombinant plants expressing such dsRNA sequences for ingestion by various plant pests, facilitating down-regulation of a gene in the genome of the pest organism and improving the resistance of the plant to the pest infestation.

The modulatory effect of dsRNA is applicable to a variety of genes expressed in the pathogens including, for example, endogenous genes responsible for cellular metabolism or cellular transformation, including house keeping genes, transcription factors and other genes which encode polypeptides involved in cellular metabolism.

As used herein, the phrase “inhibition of gene expression” or “inhibiting expression of a target gene in the cell of a pathogen” refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule. The inhibition of gene expression of the target gene in the pathogen may result in novel phenotypic traits in the pathogen.

The present invention provides in part a delivery system for the delivery of the pathogen control agents by ingestion of host cells or the contents of the cells. In accordance with another embodiment, the present invention involves generating a transgenic plant cell or a plant that contains a recombinant DNA construct transcribing the stabilized dsRNA molecules of the present invention. As used herein, the phrase “generating a transgenic plant cell or a plant” refers to the methods of employing the recombinant DNA technologies readily available in the art (e.g., by Sambrook, et al., 1989) to construct a plant transformation vector transcribing the stabilized dsRNA molecules of the present invention, to transform the plant cell or the plant and to generate the transgenic plant cell or the transgenic plant that contain the transcribed, stabilized dsRNA molecules.

The present invention alternatively provides exposure of a pathogen to the control agents of the present invention incorporated in a spray mixer and applied to the surface of a host, such as a host plant. In an exemplary embodiment, ingestion of the control agents by a pathogen delivers the control agents to the cells of the pathogen. In yet another embodiment, the RNA molecules themselves are encapsulated in a synthetic matrix such as a polymer and applied to the surface of a host such as a plant. Ingestion of the host cells by a pathogen permits delivery of the control agents to the pathogen and results in down-regulation of a target gene in the host.

It is envisioned that the compositions of the present invention can be incorporated within the seeds of a plant species either as a product of expression from a recombinant gene incorporated into a genome of the plant cells, or incorporated into a coating or seed treatment that is applied to the seed before planting. The plant cell containing a recombinant gene is considered herein to be a transgenic event.

The present invention provides in part a delivery system for the delivery of disease control agents to pathogens. The stabilized dsRNA or siRNA molecules of the present invention may be directly introduced into the cells of one or more pathogens, or introduced into an extracellular space (e.g. the plant apoplast). Methods for introduction may include direct mixing of RNA with media for the pathogen, as well as engineered approaches in which a species that is a host is engineered to express the dsRNA or siRNA. In one in vitro embodiment, for example, the dsRNA or siRNA molecules may be incorporated into, or overlaid on the top of, pathogen growth media. In another embodiment, the RNA may be sprayed onto a plant surface. In still another embodiment, the dsRNA or siRNA may be expressed by microorganisms and the microorganisms may be applied onto a plant surface or introduced into a root, stem by a physical means such as an injection. In still another embodiment, a plant may be genetically engineered to express the dsRNA or siRNA in an amount sufficient to kill the pathogens known to infect the plant.

It is also anticipated that dsRNAs produced by chemical or enzymatic synthesis may be formulated in a manner consistent with common agricultural practices and used as spray-on products for controlling plant disease. The formulations may include the appropriate stickers and wetters required for efficient foliar coverage as well as UV protectants to protect dsRNAs from UV damage. Such additives are commonly used in the bioinsecticide industry and are well known to those skilled in the art. Such applications could be combined with other spray-on insecticide applications, biologically based or not, to enhance plant protection from insect feeding damage.

The present invention also relates to recombinant DNA constructs for expression in a microorganism. Exogenous nucleic acids from which an RNA of interest is transcribed can be introduced into a microbial host cell, such as a bacterial cell or a fungal cell, using methods known in the art.

The nucleotide sequences of the present invention may be introduced into a wide variety of prokaryotic and eukaryotic microorganism hosts to produce the stabilized dsRNA or siRNA molecules. The term “microorganism” includes prokaryotic and eukaryotic microbial species such as bacteria and fungi. Fungi include yeasts and filamentous fungi, among others. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae, Actinomycetales, and Nitrobacteraceae. Among eukaryotes are fungi, such as Phycomycetes and Ascomycetes, which includes filamentous fungi such as Sclerotinia, Erysiphe, and the like, and yeast, such as Saccharomyces and Schizosaccharomyces; Basidiomycetes, such as Rhodotorula, Aureobasidium, Sporobolomyces, Phakopsora, and the like; and Oomycetes, such as Phytophthora.

D. Transgenic Plants

The present invention provides seeds and plants having one or more transgenic event. Combinations of events are referred to as “stacked” transgenic events. These stacked transgenic events can be events that are directed to controlling the same target pathogen, or they can be directed to different target pathogens or pests. In one embodiment, a seed having the ability to express a nucleic acid provided herein also has the ability to express at least one other agent, including, but not limited to, an RNA molecule the sequence of which is derived from the sequence of an RNA expressed in a target pathogen and that forms a double stranded RNA structure upon expressing in the seed or cells of a plant grown from the seed, wherein the ingestion of one or more cells of the plant by the target pathogen results in the suppression of expression of the RNA in the cells of the target pathogen.

In certain embodiments, a seed having the ability to express a dsRNA the sequence of which is derived from a target pathogen also has a transgenic event that provides herbicide tolerance. One beneficial example of a herbicide tolerance gene provides resistance to glyphosate, N-(phosphonomethyl) glycine, including the isopropylamine salt form of such herbicide.

Benefits provided by the present invention may include, but are not limited to: the ease of introducing dsRNA into the pathogen's cells, the low concentration of dsRNA which can be used, the stability of dsRNA, and the effectiveness of the inhibition. The ability to use a low concentration of a stabilized dsRNA avoids several disadvantages of anti-sense interference. The present invention is not limited to in vitro use or to specific sequence compositions, to a particular set of target genes, a particular portion of the target gene's nucleotide sequence, or a particular transgene or to a particular delivery method, as opposed to the some of the available techniques known in the art, such as antisense and co-suppression. Furthermore, genetic manipulation becomes possible in organisms that are not classical genetic models.

In practicing the present invention, it is important that the presence of the nucleotide sequences that are transcribed from the recombinant construct are neither harmful to cells of the plant in which they are expressed in accordance with the invention, nor harmful to an animal food chain and in particular humans. Because the produce of the plant may be made available for human ingestion, the down-regulation of expression of the target nucleotide sequence occurs only in the pathogen. Therefore, in order to achieve inhibition of a target gene selectively within a pathogen species that it is desired to control, the target gene should preferably exhibit a low degree of sequence identity with corresponding genes in a plant or a vertebrate animal. Preferably the degree of the sequence identity is less than approximately 80%. More preferably the degree of the sequence identity is less than approximately 70%. Most preferably the degree of the sequence identity is less than approximately 60%.

In addition to direct transformation of a plant with a recombinant DNA construct, transgenic plants can be prepared by crossing a first plant having a recombinant DNA construct with a second plant lacking the construct. For example, recombinant DNA for gene suppression can be introduced into first plant line that is amenable to transformation to produce a transgenic plant that can be crossed with a second plant line to introgress the recombinant DNA for gene suppression into the second plant line.

The present invention can be, in practice, combined with other disease control traits in a plant to achieve desired traits for enhanced control of plant disease. Combining disease control traits that employ distinct modes-of-action can provide protected transgenic plants with superior durability over plants harboring a single control trait because of the reduced probability that resistance will develop in the field.

The invention also relates to commodity products containing one or more of the sequences of the present invention, and produced from a recombinant plant or seed containing one or more of the nucleotide sequences of the present invention are specifically contemplated as embodiments of the present invention. A commodity product containing one or more of the sequences of the present invention is intended to include, but not be limited to, meals, oils, crushed or whole grains or seeds of a plant, or any food product comprising any meal, oil, or crushed or whole grain of a recombinant plant or seed containing one or more of the sequences of the present invention. The detection of one or more of the sequences of the present invention in one or more commodity or commodity products contemplated herein is defacto evidence that the commodity or commodity product is composed of a transgenic plant designed to express one or more of the nucleotides sequences of the present invention for the purpose of controlling plant disease using dsRNA mediated gene suppression methods.

D. Obtaining Nucleic Acids

The present invention provides a method for obtaining a nucleic acid comprising a nucleotide sequence for producing a dsRNA or siRNA. In one embodiment, such a method comprises: (a) probing a cDNA or gDNA library with a hybridization probe comprising all or a portion of a nucleotide sequence or a homolog thereof from a targeted pathogen; (b) identifying a DNA clone that hybridizes with the hybridization probe; (c) isolating the DNA clone identified in step (b); and (d) sequencing the cDNA or gDNA fragment that comprises the clone isolated in step (c) wherein the sequenced nucleic acid molecule transcribes all or a substantial portion of the RNA nucleotide acid sequence or a homolog thereof.

In another embodiment, a method of the present invention for obtaining a nucleic acid fragment comprising a nucleotide sequence for producing a substantial portion of a dsRNA or siRNA comprises: (a) synthesizing first and a second oligonucleotide primers corresponding to a portion of one of the nucleotide sequences from a targeted pathogen; and (b) amplifying a cDNA or gDNA insert present in a cloning vector using the first and second oligonucleotide primers of step (a) wherein the amplified nucleic acid molecule transcribes a substantial portion of the a substantial portion of a dsRNA or siRNA of the present invention.

In practicing the present invention, a target gene may be derived from a pathogen species that causes damage to the crop plants and subsequent yield losses. It is contemplated that several criteria may be employed in the selection of preferred target genes. The gene is one whose protein product has a rapid turnover rate, so that dsRNA inhibition will result in a rapid decrease in protein levels. In certain embodiments it is advantageous to select a gene for which a small drop in expression level results in deleterious effects for the pathogen. If it is desired to target a broad range of pathogen species, a gene is selected that is highly conserved across these species. Conversely, for the purpose of conferring specificity, in certain embodiments of the invention, a gene is selected that contains regions that are poorly conserved between individual species, or between the pathogen and other organisms. In certain embodiments it may be desirable to select a gene that has no known homologs in other organisms.

As used herein, the term “derived from” refers to a specified nucleotide sequence that may be obtained from a particular specified source or species, albeit not necessarily directly from that specified source or species.

In one embodiment, a gene is selected that is expressed in fungal haustoria. Targeting genes expressed in the haustorium may result in interference with a pathogen's ability to successfully colonize a host and cause disease. Target genes for use in the present invention may include, for example, those that share substantial homologies to the nucleotide sequences of known haustorial-expressed genes that encode protein components of hexose transporters.

In another embodiment, a gene is selected that is essentially involved in the growth, development, and reproduction of a pathogen. Exemplary genes include but are not limited to a β-tubulin gene. The beta-tubulin gene family encodes microtubule-associated proteins that are a constituent of the cellular cytoskeleton. Related sequences are found in such diverse organisms as Caenorhabditis elegans, and Manduca sexta.

Other target genes for use in the present invention may include, for example, those that play important roles in the viability, growth, development, reproduction and infectivity. These target genes may be one of the house keeping genes, transcription factors and the like. Additionally, the nucleotide sequences for use in the present invention may also be derived from plant, viral, bacterial or insect genes whose functions have been established from literature and the nucleotide sequences of which share substantial similarity with the target genes in the genome of a pathogen. According to one aspect of the present invention for plant disease control, the target sequences may essentially be derived from the targeted pathogen. Some of the exemplary target sequences cloned from a pathogen that encode proteins or fragments thereof which are homologues of known proteins may be found in the Sequence Listing. For instance, nucleic acid molecules from S. sclerotiorum encoding homologs of beta tubulin protein are known (e.g. SEQ ID NO:3; GenBank AAL86733).

For the purpose of the present invention, the dsRNA or siRNA molecules may be obtained by polymerase chain (PCR™) amplification of a target gene sequences derived from a gDNA or cDNA library or portions thereof. The DNA library may be prepared using methods known to the ordinary skilled in the art and DNA/RNA may be extracted. Genomic DNA or cDNA libraries generated from a pathogen may be used for PCR™ amplification for production of the dsRNA or siRNA.

The target genes may be then be PCR™ amplified and sequenced using the methods readily available in the art. One skilled in the art may be able to modify the PCR™ conditions to ensure optimal PCR™ product formation. The confirmed PCR™ product may be used as a template for in vitro transcription to generate sense and antisense RNA with the included minimal promoters.

The present inventors contemplate that nucleic acid sequences identified and isolated from any fungal or oomycete species may be used in the present invention for control of plant disease. In one aspect of the present invention, the nucleic acid may be derived from a rust fungus species. Specifically, the nucleic acid may be derived from Phakopsora pachyrizi, the causal agent of Asian soy rust The isolated nucleic acids may be useful, for example, in identifying a target gene and in constructing a recombinant vector that produce stabilized dsRNAs or siRNAs of the present invention for protecting plants from Asian soy rust.

Therefore, in one embodiment, the present invention comprises isolated and purified nucleotide sequences that may be used as plant disease control agents. The isolated and purified nucleotide sequences may comprise those as set forth in the sequence listing.

The nucleic acids from S. sclerotiorum that may be used in the present invention may also comprise isolated and substantially purified Unigenes and EST nucleic acid molecules or nucleic acid fragment molecules thereof. EST nucleic acid molecules may encode significant portions of or indeed most of the polypeptides. Alternatively, the fragments may comprise smaller oligonucleotides having from about 15 to about 250 nucleotide residues, and more preferably, about 15 to about 30 nucleotide residues. Alternatively, the nucleic acid molecules for use in the present invention may be from cDNA libraries from a fungus of interest.

Nucleic acid molecules and fragments thereof from pathogen species may be employed to obtain other nucleic acid molecules from other species for use in the present invention to produce desired dsRNA and siRNA molecules. Such nucleic acid molecules include the nucleic acid molecules that encode the complete coding sequence of a protein and promoters and flanking sequences of such molecules. In addition, such nucleic acid molecules include nucleic acid molecules that encode for gene family members. Such molecules can be readily obtained by using the above-described nucleic acid molecules or fragments thereof to screen cDNA or genomic DNA libraries. Methods for forming such libraries are well known in the art.

As used herein, the phrase “coding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

The term “recombinant DNA” or “recombinant nucleotide sequence” refers to DNA that contains a genetically engineered modification through manipulation via mutagenesis, restriction enzymes, and the like.

For many of the pathogens that are potential targets for control by the present invention, there may be limited information regarding the sequences of most genes or the phenotype resulting from mutation of particular genes. Therefore, the present inventors contemplate that selection of appropriate genes from pathogens for use in the present invention may be accomplished through use of information available from study of the corresponding genes in a model organism such in Saccharomyces cerevisiae, or even in a nematode species, in an insect species, or in a plant species, in which the genes have been characterized. In some cases it will be possible to obtain the sequence of a corresponding gene from a target pathogen by searching databases such as GenBank using either the name of the gene or the sequence from, for example, Drosophila, another insect, a nematode, or a plant from which the gene has been cloned. Once the sequence is obtained, PCR™ may be used to amplify an appropriately selected segment of the gene in the pathogen for use in the present invention.

In order to obtain a DNA segment from the corresponding gene in a fungal species, PCR™ primers may be designed based on the sequence as found in another organism from which the gene has been cloned. The primers are designed to amplify a DNA segment of sufficient length for use in the present invention. DNA (either genomic DNA or cDNA) is prepared from the pathogen, and the PCR™ primers are used to amplify the DNA segment. Amplification conditions are selected so that amplification will occur even if the primers do not exactly match the target sequence. Alternately, the gene (or a portion thereof) may be cloned from a gDNA or cDNA library prepared from the pathogen species, using the known gene as a probe. Techniques for performing PCR™ and cloning from libraries are known. Further details of the process by which DNA segments from target pathogen species may be isolated based on the sequence of genes previously cloned from other species are provided in the Examples. One of ordinary skill in the art will recognize that a variety of techniques may be used to isolate gene segments from plant pathogenic microorganisms that correspond to genes previously isolated from other species.

### EXAMPLES

The inventors herein have identified a means for controlling plant pathogen infection by providing a double stranded ribonucleic acid molecules in pathogen host tissues. Double stranded ribonucleic acid molecules that function upon ingestion by the pathogen to inhibit a biological function in the pathogen, may result, for example, in one or more of the following attributes: reduction in viability of the pathogen, death of the pathogen, inhibition of differentiation and development of the pathogen, absence of or reduced capacity for ion regulation and transport, maintenance of cell membrane potential, development of appressoria or haustoria, penetration of host, amino acid biosynthesis, amino acid degradation, development and differentiation, cell division, energy metabolism, respiration, apoptosis, and any component of a eukaryotic cells' cytoskeletal structure, such as, for example, actins and tubulins. Any one or any combination of these attributes can result in an effective inhibition of plant infection or colonization, and in the case of a plant pathogenic fungus or oomycete, inhibition of plant disease, and/or reduction in severity of disease symptoms.

Similar analyses of plant-pathogen control by dsRNA-mediated gene suppression can be performed in corn, cotton, canola, wheat and other important field and vegetable crops. The Examples set forth herein below are illustrative of the invention when applied to a single pathogen. However, the skilled artisan will recognize that the methods, formulae, and ideas presented in the Examples are not intended to be limiting, and are applicable to all fungal or oomycete plant pathogenic species that form a nutritional relationship with a plant that contains a sufficient amount of an inhibitory agent consisting at least of one or more double stranded RNA molecules exemplified herein intended to suppress some essential feature about or function within the pathogen.

### Example 1

### Analysis of in Planta dsRNA Uptake by Fungi

The ability of heterotrophic fungi to take up dsRNA molecules of various design (full ORF length, segments of ORFs, or “diced” to make siRNA) is tested both in artificial growth media and in planta. To test in planta uptake of a heterotrophic fungus, vectors encoding dsRNA designed to suppress S. sclerotiorum genes are designed. The vectors are transformed into both Arabidopsis thaliana and Soy (Glycine max). S. sclerotiorum is inoculated to transgenic soy or Arabidopsis plants and both the infection progress of the fungus and fungal gene suppression is evaluated as measure of dsRNA-mediated gene suppression. The genes chosen for the analysis include essential genes (tubulin, vATPase) and PacI, a gene essential for virulence (Rollins, 2003).

The ability of biotrophic pathogens to take up dsRNA or siRNA molecules which can suppress essential genes and thus provide disease resistance is tested in a model plant-pathogen system. Arabidopsis plants are transformed with constructs designed to suppress powdery mildew (Erysiphe cichoracearum) genes. Biotrophic pathogens such as E. cichoracearum produce haustoria (specialized feeding structures) in plant cells which could take up dsRNA or siRNA molecules thus allowing gene suppression. Constructs encoding dsRNA molecules designed to suppress essential genes (tubulin, vATPase) and a MAP kinase gene (if present) involved in pathogenicity are tested. The strategy used on this model biotrophic pathogen system will also be applied towards the causal agent of Asian soy rust (Phakopsora pachyrizi), an economically important biotrophic soy pathogen.

### Example 2

### Analysis of Secreted Fungal dsRNAse Activity

To test the ability of heterotrophic fungi to take up dsRNA molecules from artificial growth media, the degree of dsRNAse activity secreted by Sckrotinia sclerotiorum (causal agent of soy white mold), and Neurospora crassa, a model Ascomycete fungus, was determined. Fungi were grown in stationary culture until significant fungal mats were observed (5-14 days). Cell-free aliquots of growth media were incubated in the presence of dsRNA molecules. The dsRNA was designed to suppress vATPase of Western corn rootworm (Diabrotica virgifera). This dsRNA was not designed to suppress a fungal gene; in these assays it was used solely to test for secreted dsRNAse activity. Both N. crassa and S. sclerotiorum secrete dsRNAse activities sufficient to degrade the tested dsRNA molecule. Incubated dsRNA were run on standard non-denaturing TBE-agarose gels. These gels do not have the resolving power to detect siRNA-like (18-23 by dsRNA) sequences that could have been produced by the observed secreted dsRNAse activities produced by either fungus. dsRNA molecules to be tested include full length ORFs, segments of ORFs, and dsRNA molecules of an siRNA-like size. Further analysis is performed to determine if siRNA-like molecules are produced by the secreted dsRNAse of each fungus.

### Example 3

### Identification of Target Nucleotide Sequences For Preparation of dsRNA Useful for Controlling Fungi and Preparation of Plant Transformation Vectors

Plant expression vectors encoding dsRNA molecules designed to suppress fungal gene expression were designed, for use in transforming Arabidopsis and soybean. The heterotrophic fungus S. sclerotiorum and the biotrophic fungus Erysiphe cichoracearum (powdery mildew) were chosen for this analysis. Biotrophic fungi produce specialized structures in plant cells, called haustoria, that may be able to take up dsRNA or siRNA molecules, thus allowing suppression of gene expression. Constructs encoding dsRNA molecules designed to suppress essential genes (e.g. tubulin, vATPase), and a phosphatase that regulates MAP Kinase activity involved in pathogenicity were designed. E. cichoracearum is considered a model biotrophic pathogen; a similar strategy may also be employed to suppress Asian soy rust (causal agent Phakopsora pachyrizi), an economically important biotrophic soy pathogen.

A. Design of dsRNA Targeting Beta Tubulin Expression

Tubulin proteins are important structural components of many cellular structures in all eukaryote cells and principally in the formation of microtubules Inhibition of microtubule formation in cells results in catastrophic effects including interference with the formation of mitotic spindles, blockage of cell division, and the like. Therefore, suppression of tubulin protein formation may be a useful target for double stranded RNA mediated inhibition.

Degenerate PCR primers were designed to clone fungal tubulin genes by PCR. PCR primer prJWP296 (SEQ ID NO:1; AAYAAYTGGGCIAARGGICA) is an 8-fold degenerate forward primer based on the conserved tubulin sequence NNWAKGH (SEQ ID NO:36) found around residue 99 of the alignment of the genes noted below. PCR primer prJWP297 (SEQ ID NO:2; TCCATYTCRTCCATNCCYTC) is a 32-fold degenerate reverse primer based on the fungal tubulin sequence EGMDEME (SEQ ID NO:37) based on the amino acid sequence found around residue 401 of the alignment of the following listed genes. The fungal tubulin sequences used to design these primers include S. sclerotiorum beta tubulin (SEQ ID NO:3; GenBank accession AAL86733); Erysiphe pisi tubulin beta chain (SEQ ID NO:4 Swissprot accession P40905); Blumeria graminis beta tubulin (SEQ ID NO:5; Swissprot accession P16040); Gibberella fujikoroi beta tubulin (SEQ ID NO:6; GenBank accession AAB18275); Gibberella zeae beta tubulin (SEQ ID NO:7; GenBank accession AAP68979); Aspergillus nidulans beta tubulin (SEQ ID NO:8; GenBank accession XP 405319); N. crassa tubulin beta chain (SEQ ID NO:9; GenBank accession XP_{—}323373); A. flavus tubulin beta chain (SEQ ID NO:10; Swissprot accession P22012); Magnaporthe grisea (SEQ ID NO:11; GenBank accession EAA48946); Epichloe typhina tubulin beta chain (SEQ ID NO:12; Swissprot accession P17938); Colletotrichum graminicola tubulin beta chain 2 (SEQ ID NO:13; GenBank accession JQ0423); Botryotinia fuckeliana tubulin beta chain (SEQ ID NO:14; Swissprot accession P53373); and Leptosphaeria maculans beta tubulin (SEQ ID NO:15; GenBank accession AAF66992).

B. Design of dsRNA Targeting vATPase Expression

Energy metabolism within subcellular organelles in eukaryotic systems is an essential function. Vacuolar ATP synthases are involved in maintaining sufficient levels of ATP within vacuoles, maintaining an electrochemical gradient across plant cell membranes, and for vacuolar function including maintenance of cell turgor and transport and storage of various ions and metabolites. Therefore, vacuolar ATP synthases may be a useful target for double stranded RNA mediated inhibition. Degenerate PCR primers were designed to clone fungal vATPase genes by PCR. PCR primer prJWP298 (SEQ ID NO:16; ATHCARGTITAYGARGARAC) is a 48-fold degenerate forward primer based on the conserved sequence IQVYEET (SEQ ID NO:38) starting around amino acid residue 70 of the alignment of the genes noted below. prJWP299 (SEQ ID NO:17; CCYTGRTCIGCIGGCATYTC) is a 16-fold degenerate reverse primer based on the amino acid sequence EMPADQG (SEQ ID NO:39) found around amino acid residue 374 of the alignment of genes noted below. The aligned vATPase sequences included G. zeae vATPase (SEQ ID NO:18); A. oryzae vATPase SEQ ID NO:19); Saccharomyces cerevisiae vATPase (SEQ ID NO:20); S. pastorianus vATPase (SEQ ID NO:21); N. crassa vATPase (SEQ ID NO:22); Candida albicans vATPase (SEQ ID NO:23).

C. Design of dsRNA Targeting PacI Phosphatase

The PacI Tyr/Thr phosphatase regulates MAP kinase activity and is required for sclerotia development and full virulence in S. sclerotiorum (Rollins 2003), making it a target for dsRNA-mediated inhibition of expression. Degenerate PCR primers were designed to clone the S. sclerotiorum PacI gene (SEQ ID NO:24) by PCR. prJWP292, a 32-fold degenerate 20-mer (SEQ ID NO:25; TTYGAYCAYATHTGYGA) forward primer, was designed based on the sequence FDHICE (SEQ ID NO:40) found around amino acid residue of the PacI protein. prJWP293, a 64-fold degenerate reverse primer (TCYTCRTCYTCYTCRTCYTT; SEQ ID NO:26) was designed based on amino acid residues KDEEDED (SEQ ID NO:41) found around residue 585. prJWP294, an eight-fold degenerate forward primer (SEQ ID NO:27; CCTATGCCICARCAYCARTA) was based on the sequence PMPQHQY (SEQ ID NO:42) found around residue 304. prJWP295, a 24-fold degenerate reverse primer (SEQ ID NO:28; TTYTCDATCCAIGCYTCYTC), was designed based on the sequence EEAWIEN (SEQ ID NO:43) found around residue 557 of the predicted PacI protein sequence. The PacI nucleic acid sequence is found at SEQ ID NO:29 (GenBank AY005467).

D. Other Fungal Targets for dsRNA-Mediated Inhibition of Expression

Other fungal genes may be selected as targets for dsRNA-mediated inhibition of expression. For instance, a gene required for successful infection, penetration, or mycelial growth may be selected. Alternatively, fungal genes may be selected based on their expression pattern in planta. For instance, a gene that is up-regulated during fungal growth in planta may be chosen. A number of such genes have been identified, including Magnaporthe grisea hydrophobin (Matsumura et al., 2003), a haustorial hexose transporter HXT1 from the rust fungus Uromyces Jabae (Voegele et al., 2001), an amino acid transporter (e.g. from U. fabae; Hahn et al., 1997), and FIS1, a probable aldehyde dehydrogenase (Deutschle et al., 2001). A transcript profiling analysis may be carried out to identify such genes, for instance those genes largely or specifically expressed during infection or growth in planta. Another possible target for dsRNA-mediated inhibition is the set of genes that interact with plant avirulence genes.

E. Preparation of Plant Transformation Vectors

Plant transformation vectors were prepared using the S. sclerotiorum PacI, tubulin, and vATPase genes cloned as described. pMON96284 comprises a cassette consisting of the enhanced CaMV 35S promoter linked to a region from the S. sclerotiorum PacI gene (SEQ ID NO:30) and a transcriptional terminator. pMON96286 comprises a cassette consisting of the enhanced CaMV 35S promoter linked to a region from the S. sclerotiorum tubulin gene (SEQ ID NO:31) and a transcriptional terminator. pMON96289 comprises a cassette consisting of the enhanced CaMV 35S promoter linked to a region from the S. sclerotiorum vATPase gene (SEQ ID NO:32) and a transcriptional terminator. Each of these vectors is designed for Agrobacterium-mediated transformation, and Arabidopsis plants were transformed with these constructs by standard methods. The presence and integrity of a desired construct in transformed plant cells is confirmed by Southern blot analysis (of 2-4 independent tranformants), and northern analysis is performed to test whether the target gene is suppressed. Plants are also tested for resistance to disease caused by S. sclerotiorum due to suppression of the genes of interest.

| Vector | Sequence Expressed as dsRNA | SEQ ID NO |
|---|---|---|
| pMON96284 | Pac1 | SEQ ID NO: 30 |
| pMON96286 | Beta-tubulin | SEQ ID NO: 31 |
| pMON96289 | V-ATPase | SEQ ID NO: 32 |

### Example 4

Analysis of dsRNA-Mediated Fungal Gene Suppression by Transient Expression in Tobacco

pMON96284, pMON96286, and pMON96289 were infiltrated into attached or detached leaves of Nicotiana benthamiana. Attached infiltrated leaves were detached from plants 2-4 days post infiltration and placed in large petri plates containing 3 Whatman #1 filter papers saturated with distilled water. Agar plugs containing S. sclerotiorum were placed on the leaves, and the edges of the plates were sealed and placed in a Percival incubator set at 22° C. with a 12 hour light cycle. Lesion growth was followed over a period of 4-7 days. No inhibition of fungal lesion expansion was observed.

### Example 5

Analysis of Fungal Growth Inhibition by In Vitro Uptake of dsRNA Designed to Inhibit Fungal Gene Expression

The ability of S. sclerotiorum to take up dsRNA molecules from liquid growth medium and result in gene suppression was studied. dsRNA was prepared from vectors expressing gene fragments of the tubulin, PacI, and vATPase genes. The experiments were performed in 96 well plates with each well containing 150 μl of potato dextrose broth supplemented with 0, 1, 10, or 100 ppm dsRNA homologous to PacI, tubulin, vATPase of S. sclerotiorum, or GFP (sdRNA control). Mycelial-agar plugs of S. sclerotiorum were inoculated to each well and growth was monitored for up to 2 days. No inhibition of fungal growth was observed. siRNA molecules designed against the above targets may also be tested for in vitro gene suppression effects.

### Example 6

### Soy Rust vATPase as a Target for dsRNA-Mediated Gene Suppression

Soy rust (P. pachyrizi) vATPase gene sequences (A subunit and B subunit) were identified in NCBI Genbank. The sequences were used to direct the PCR synthesis of vATPase gene fragments which will be used to construct soy transformation vectors expressing vATPase dsRNA molecules. Plants expressing these dsRNA molecules will be tested for evidence of gene suppression. A 501 nucleotide segment (SEQ ID NO:33), a 486 nucleotide segment (SEQ ID NO:34), and an 819 nucleotide segment (SEQ ID NO:35), each derived from P. pachyrizi vATPase sequence, were utilized to design primers for PCR-based synthesis, following modification to remove regions at least 21 nucleotides in length with similarity to sequences found in other organisms, including humans. For synthesis of B subunit, top and bottom strands were synthesized as 40-mer primers with 20 nt overlaps. The A subunit gene was cloned similarly, with 42-mer primers covering both strands with 21 nt overlaps.

A two step PCR protocol was followed. Primers were suspended and pooled at a concentration of 1 μM each. 1 μl of the pooled primers was run with the following temperature parameters: 1 cycle 93° C., 2 minutes; 8 cycles 93° C.-50° C.-68° C. (30″; 30″; 1′); 8 cycles 93° C.-45° C.-68° C. (30″; 30″; 1′); 8 cycles 93° C.-42° C.-68° C. (30″; 30″; 1′); 8 cycles 93° C.-40° C.-68° C. (30″; 30″; 1′); 8 cycles 93° C.-39° C.-68° C. (30″; 30″; 1′); 4° C. hold. Enzymes used were Roche Faststart Hi-Fidelity; Extaq; and Invitrogen Pfx Platinum. Following this PCR step, 1 μl of the PCR product, or alternatively 4 μl of a 1:10 or 1:50 dilution were used in a PCR cloning experiment with parameters as described above, but with half of the number cycles at each step. Products were run on an agarose gel, cut out, eluted with Qiaquick (Qiagen,), concentrated with ethanol precipitation, ligated into IpCR-BluntII-TOPO vector by blunt end ligation, and transformed into competent TOP10 cells. Transformed cells were plated on Kanamycin-containing LB plates, single colony purified, and grown in liquid Kanamycin-containing media for 16 hours. Plasmid DNA was prepared by Qiagen miniprep, and confirmed by sequencing and restriction digest to confirm insert size.

### Example 7

### Evaluation of dsRNA to Target Genes in Oomycete and Fungal Pathogens

Fungal or Oomycete transformation constructs are prepared using the target genes of interest of Examples 3, 5, and 6. Stable transformants are obtained. The presence and integrity of the desired construct is validated by Southern analysis (2-4 independent transformants, each), and northern analysis is performed to test for gene suppression. Control strains are untransformed fungal strains used as transformation recipients. Relative gene expression controls include, for example, tubulin, and GPD (glyceraldehyde-p-dehydrogenase). siRNA analysis of the target gene may be performed, as well as in vitro growth studies to confirm that the transformed fungal strains have no growth rate or morphological defects that would affect pathogenicity, if appropriate. Biochemical assays to detect changes in activity of a gene may also be performed if appropriate. Pathogenicity assays on an appropriate plant host arc performed, using multiple independently transformed fungal strains. Control pathogen strains transformed with a gene construct expressing an dsRNA unrelated to fungal growth or pathogenicity are also constructed, to compare gene suppression and effects on pathogenicity, and to confirm that suppression is gene specific.

### Example 8

### Design of dsRNA Molecules for Enhanced Gene Suppression Activity

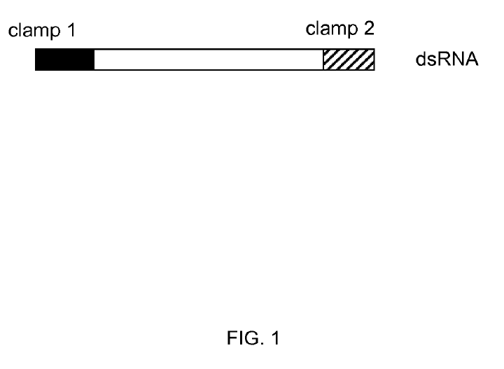

A method of stabilizing dsRNA molecules would be to “clamp” the ends of the molecules using GC rich sequences. An example of such a dsRNA molecule is a linear complementary RNA molecule either assembled from two constructs, or expressed from two promoters on the same construct. (FIG. 1). In FIG. 1, Clamp1 and Clamp2 represent GC rich dsRNA regions (i.e. little or no A or T coding nucleotides) that arc not complementary to each other, and are unrelated to the gene of interest, or related if such a GC rich region exists within the gene. The GC rich clamp regions serve to thermodynamically stabilize the dsRNA molecules which may increase gene silencing. The clamps can be of varying sizes which can be determined empirically, but are probably from 25-100 by in length.

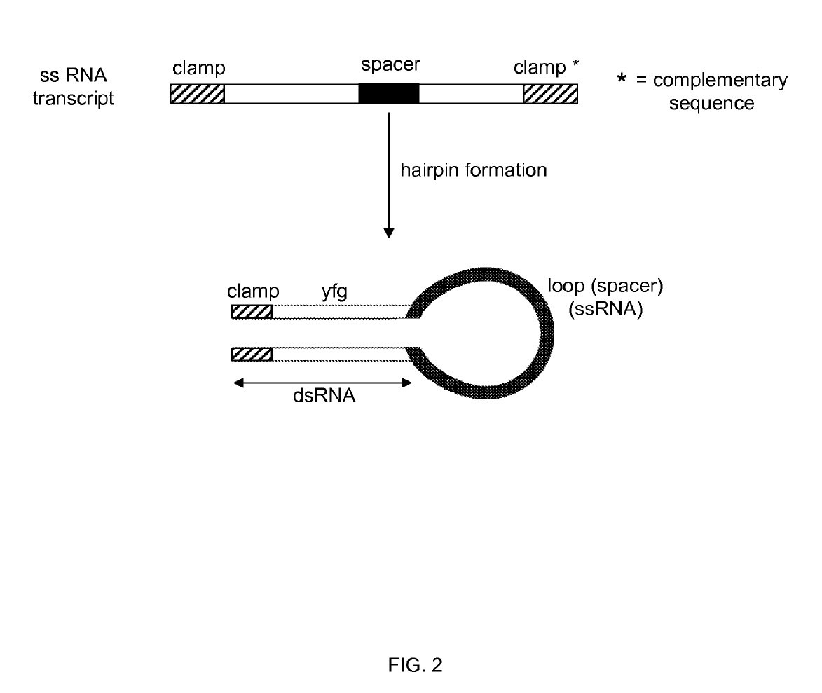

A clamping strategy for hairpin dsRNA molecules derived from a single expression cassette could utilize either a single clamp to hold the free ends together, as shown in FIG. 2. In this case, the ends of the ssRNA molecule are complementary and serve to stabilize the dsRNA region of the hairpin along with the complementary strands of the gene of interest which are separated by a spacer region.

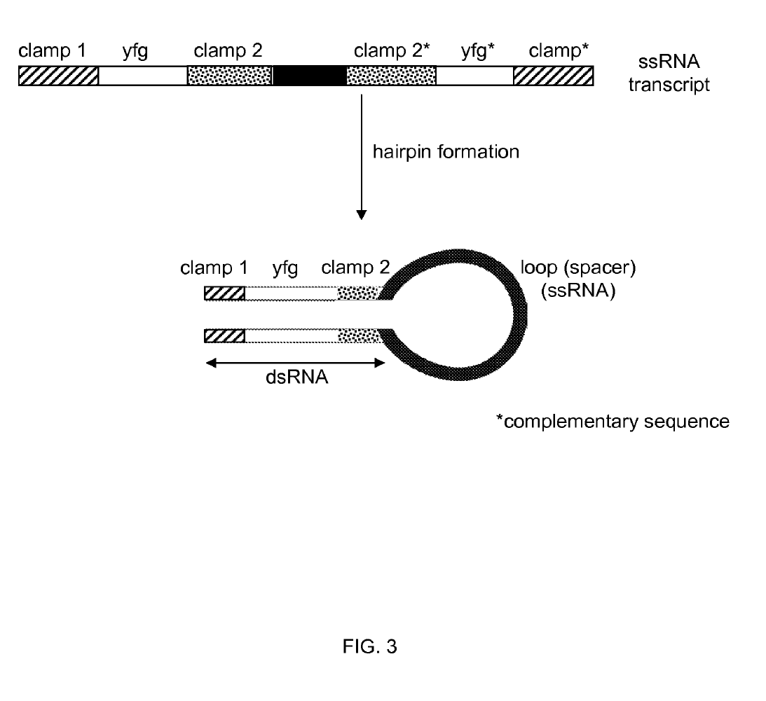

A similar strategy employs two clamps which are not complementary to each other, that clamp the free ends of the hairpin and the area adjacent to the spacer/hairpin region of the molecule (FIG. 3). In this case, two complementary clamps are formed from the two complementary sequences for each clamp region. The two clamps are not complementary to each other in this example.

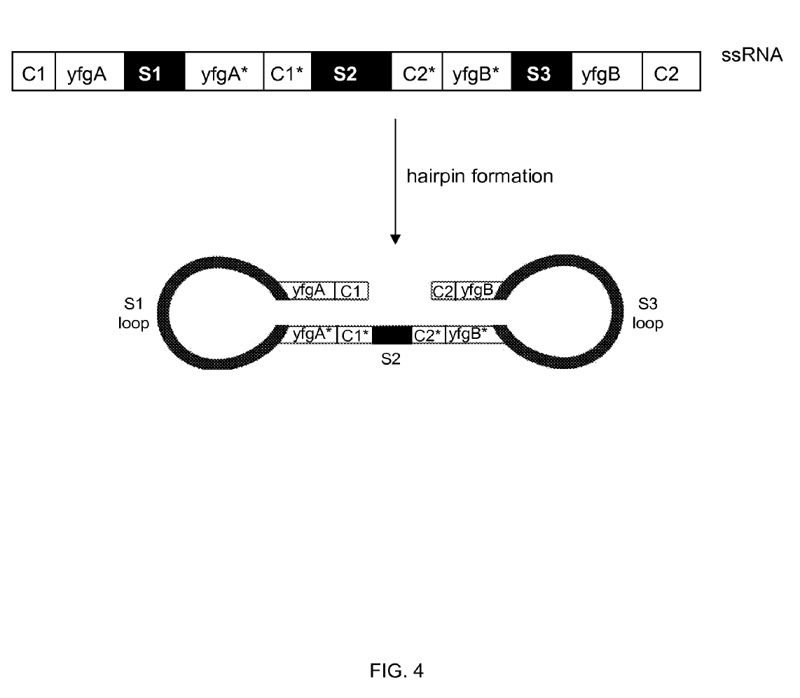

FIG. 4 illustrates an example wherein two regions of a given gene, or two independent genes, are separated by a spacer region, with a clamp on either end. The resulting ssRNA folds to form a double hairpin molecule clamped at either end.

### Example 9

### Transgenic Plant Transformation and Bioassays

Briefly, the sequence encoding a dsRNA construct as described above is linked at the 5′ end to a sequence consisting of a 35S or other heterologous promoter, optionally operably linked to an intron and at the 3′ end to a transcription termination and polyadenylation sequence. This expression cassette is placed downstream of a glyphosate selection cassette. These linked cassettes are then placed into an Agrobacterium tumefaciens plant transformation functional vector, used to transform tobacco, Arabidopsis, or soy tissue to glyphosate tolerance, and events are selected, regenerated, and transferred to soil.

### Example 10

### Implementing Pathogen Suppression Using a ta-siRNA Mediated Silencing Method

An alternative method to silence genes in a plant pathogen uses the recently discovered class of trans-acting small interfering RNA (ta-siRNA) (Dalmay et al., 2000; Mourrain et al., 2000; Peragine et al., 2004; Vazquez et al., 2004; Yu et al., 2003). ta-siRNA are derived from single strand RNA transcripts that are targeted by naturally occurring miRNA within the cell. Methods for using microRNA to trigger ta-siRNA for gene silencing in plants are described in U.S. Provisional Patent Application Ser. No. 60/643,136, incorporated herein by reference in its entirety. At least one fungal or oomycete specific miRNA expressed in a plant pathogen of interest is identified. This pathogen specific miRNA is then used to identify at least one target RNA transcript sequence complementary to the miRNA that is expressed in the cell. The corresponding target sequence is a short sequence of no more than 21 contiguous nucleotides that, when part of a RNA transcript and contacted by its corresponding miRNA in a cell type with a functional RNAi pathway, leads to slicer-mediated cleavage of said transcript. Once miRNA target sequences are identified, at least one miRNA target sequence is fused to a second sequence that corresponds to part of a pathogen gene that is to be silenced using this method. For example, the miRNA target sequence(s) is fused to a nucleotide segment of a gene of interest, such as a sequence of vacuolar ATPase (V-ATPase) gene. The miRNA target sequence can be placed at the 5′ end, the 3′ end, or embedded in the middle of the target sequence. It may be preferable to use multiple miRNA target sequences corresponding to multiple miRNA genes, or use the same miRNA target sequence multiple times in the chimera of the miRNA target sequence and the target gene sequence. The target gene sequence can be of any length, with a minimum of 21 bp.

The chimera of the miRNA target sequence(s) and the target gene sequence is expressed in plant cells using any of a number of appropriate promoter and other transcription regulatory elements, as long as the transcription occurs in cell types subject to infection and/or colonization by the pathogen.

This method may have the additional advantage of delivering longer RNA molecules to the target pathogen. Typically, dsRNA's produced in plants are rapidly processed by Dicer into short RNA's that may not be effective when fed exogenously to some pathogens. In this method, a single strand transcript is produced in the plant cell, taken up by the pathogen, and converted into a dsRNA in the pathogen cell where it is then processed into ta-siRNA capable of post-transcriptionally silencing one or more genes in one or more target pathogens.

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### REFERENCES

The following references, to the extent that they provide exemplary procedural or other details supplementary to those set forth herein, are specifically incorporated herein by reference.
Altschul et al., J. Mol. Biol. 215:403-410 (1990).
Bevan Nature 304:184-187 (1983).
Botstein et al., Gene, 8:17-24 (1979.
Brake et al., Proc. Natl. Acad. Sci. USA, 81:4642-4646 (1984).
Brutlag, et al., Comp. Chem. 17: 203-207 (1993).
Busk Plant J. 11:1285-1295 (1997).
Chicas, et al., Nucl Acids Res. 32:4237-43 (2004).
Cottrell, and Doering, Trends Microbiol. 11: 37-43 (2003).
Dalmay et al., Cell 101:543-553, (2000).
Dellaporta et al., Stadler Symposium 11:263-282 (1988).
Deutschle et al., Plant J. 27:345-56 (2001).
Dodds et al., Plant Cell 16:755-768 (2004).
Elbashir et al., Genes & Devel., 15:188-200 (2002).
Hahn et al., Mol. Plant. Microbe Interact. 10:438-45. (1997).
Hamilton and Baulcombe, Science, 286:950-952 (1999).
Haymes et al., Nucleic Acid Hybridization, A Practical Approach, IRL Press, Washington, D.C. (1985).
Herrera-Estrella Nature 303:209-213 (1983).
Ikatu et al., Bio/Technol. 8:241-242 (1990)
Jefferson et al., EMBO J. 6:3901-3907 (1987).
Kadotani, et al., Mol Plant Microbe Interact. 16:769-76 (2004).
Kaeppler et al., Plant Cell Reports 9: 415-418 (1990).
Katz et al., J. Gen. Microbiol. 129:2703-2714 (1983)
Kennerdell et al., Cell 95:1017-1026 (1998).
Klee, Bio/Technol. 3:637-642 (1985).
Lindbo & Dougherty, Ann. Rev. Phytopathol. 43:191-204 (2005).
Matsumura et al., Proc Natl Acad Sci (USA) 100:15718-15723 (2003).
Mendgen and Hahn, Trends in Plant Sciences 7:352-356 (2002).
Mendgen and Hahn, Trends Pl. Sci 7:352-356 (2002).
Mourrain et al., Cell 101:533-542, (2000).
Mouyna et al., FEMS Microbiology Letters 237: 317-24 (2004).
Myanohara et al., Proc. Natl. Acad. Sci. USA, 80:1 (1983).
Odell et al., Nature 313:810-812 (1985).
Orr-Weaver et al., Meth. Enzymol., 101:228-245 (1983).
Ow et al., Science 234:856-859 (1986)
Peragine et al., Genes and Devel., 18:2368-2379, (2004).
Poplawski et al., Curr Genet. 32:66-72 (1997).
Potrykus, Mol. Gen. Genet. 199:183-188 (1985).
Rajagopal et al., J. Biol. Chem. 277: 46849-46851 (2002).
Raponi, and Arndt. Nucl. Acids Res. 31:4481-89 (2003).
Reese, and Doering. Mol. Microbiol. 50:1401-09 (2003).
Rine et al., Proc. Natl. Acad. Sci. (USA), 80:6750 (1983).
Rollins, J. A. Mol. Plant-Microbe Interact. 16:785-95 (2003).
Sambrook et al., Molecular Cloning, A Laboratory Manual, 2nd Edition, Cold Spring Harbor Press, Cold Spring Harbor, N.Y., (1989).
Schultheiss, H., et al., Pl. Physiol. 128:1447-1454 (2002).
Stahl et al., BMC Biotechnology, 4:31 (2004).
Stinchcomb et al., J. Mol. Biol., 158:157 (1982).
Sutcliffe et al., Proc. Natl. Acad. Sci. (U.S.A.) 75:3737-3741 (1978).
U.S. patent application Ser. No. 10/465,800
U.S. Patent Application Publication Nos. US 2002/0048814A1, 2003/0175965, 2003/0018993A1, 2003/0150017, and 2003/0061626
U.S. Pat. Nos. 5,107,065, 5,759,829, 5,283,184, 5,231,020, 6,506,559, and 6,326,193
Van Heeke and Schuster, J. Biol. Chem. 264:5503-5509 (1989).
Vazquez et al., Mol Cell 16:69-79, (2004).
Voegele et al., Proc Natl Acad Sci (USA); 98:8133-8138 (2001).
WO 2005/071091
WO 99/49029
WO 99/53050
WO94/01550
WO98/05770
Yu et al., Mol Plant Microbe Interact 16:206-216, (2003).
Zukowsky et al., Proc. Natl. Acad. Sci. (U.S.A. 80:1101-1105 (1983.

What is claimed is:

1. An isolated polynucleotide selected from the group consisting of:
(a) a fragment of at least 21 contiguous nucleotides of a nucleic acid sequence of SEQ ID NOs:3-15; SEQ ID NOs:18-23, SEQ ID NO:29, or SEQ ID NOs:33-35 wherein uptake by a fungal or oomycete plant pathogen of a double stranded ribonucleotide sequence comprising at least one strand that is complementary to said fragment inhibits the growth of said pathogen; and
(b) a complement of the sequence of (a).

2. The isolated polynucleotide of claim 1, defined as operably linked to a heterologous promoter.

3. The isolated polynucleotide of claim 1, defined as comprised on a plant transformation vector.

4. A double stranded ribonucleotide sequence produced from the expression of a polynucleotide according to claim 1, wherein the taking up of said ribonucleotide sequence by a fungal or oomycete plant pathogen inhibits the growth of said pathogen.

5. The double stranded ribonucleotide sequence of claim 4, defined as produced by preparing a recombinant polynucleotide sequence comprising a first, a second and a third polynucleotide sequence, wherein the first polynucleotide sequence comprises the isolated polynucleotide of claim 1, wherein the third polynucleotide sequence is linked to the first polynucleotide sequence by the second polynucleotide sequence, and wherein the third polynucleotide sequence is substantially the reverse complement of the first polynucleotide sequence such that the first and the third polynucleotide sequences hybridize when transcribed into a ribonucleic acid to form the double stranded ribonucleotide molecule stabilized by the linked second ribonucleotide sequence.

6. The double stranded ribonucleotide sequence of claim 4, wherein the taking up of the polynucleotide sequence by the pathogen inhibits the expression of a nucleotide sequence substantially complementary to said polynucleotide sequence.

7. A cell transformed with the polynucleotide of claim 1.

8. The cell of claim 7, defined as prokaryotic cell.

9. The cell of claim 7, defined as a eukaryotic cell.

10. The cell of claim 7, defined as a plant cell.

11. A plant transformed with the polynucleotide of claim 1.

12. A seed of the plant of claim 11, wherein the seed comprises the polynucleotide.

13. The plant of claim 11, wherein said polynucleotide is expressed in the plant cell as a double stranded ribonucleotide sequence.

14. The plant of claim 13, wherein the pathogen is selected from the group consisting of ascomycetes, basidiomycetes, deuteromycetes, and oomycetes.

15. The plant of claim 13, wherein the taking up of the pathogen inhibitory amount of the double stranded ribonucleotide sequence inhibits growth of the pathogen.

16. A commodity product produced from a plant according to claim 11, wherein said commodity product comprises a detectable amount of the polynucleotide of claim 1 or a ribonucleotide expressed therefrom.

17. A method for controlling fungal or oomycete plant disease comprising providing an agent comprising a double stranded ribonucleotide sequence that functions upon being taken up by the pathogen to inhibit a biological function within said pathogen.

18. A method for controlling fungal or oomycete plant disease comprising providing an agent comprising a first polynucleotide sequence that functions upon being taken up by the pathogen to inhibit a biological function within said pathogen, wherein said polynucleotide sequence exhibits from about 95 to about 100% nucleotide sequence identity along at least from about 19 to about 25 contiguous nucleotides to a coding sequence derived from said pathogen or its host plant and is hybridized to a second polynucleotide sequence that is complementary to said first polynucleotide sequence, and wherein said coding sequence derived from said pathogen or host is selected from the group consisting of SEQ ID NOs:3-15; SEQ ID NOs:18-23, SEQ ID NO:29, or SEQ ID NO:s:33-35 and the complements thereof.

19. The method of claim 18, wherein said pathogen is an ascomycete, a basidiomycete, a deuteromycete, or an oomycete.

20. A method for controlling a fungal or oomycete plant disease comprising providing in the host plant of a fungal or oomycete plant pathogen a transformed plant cell expressing a polynucleotide sequence according to claim 1, wherein the polynucleotide is expressed to produce a double stranded ribonucleic acid that functions upon being taken up by the pathogen to inhibit the expression of a target sequence within said pathogen and results in decreased growth, in or on the host of the pathogen, relative to a host lacking the transformed plant cell.

21. The method of claim 20, wherein the pathogen exhibits decreased growth following infection of the host plant.

22. The method of claim 20, wherein the target sequence encodes a protein, the predicted function of which is selected from the group consisting of: ion regulation and transport, enzyme synthesis, nutrient assimilation, viability of the pathogen, sexual reproduction by the pathogen, maintenance of cell membrane potential, amino acid biosynthesis, amino acid degradation, development and differentiation, infection, penetration, development of appressoria or haustoria, mycelial growth, fruiting body growth; sporulation; melanin synthesis, toxin synthesis, siderophore synthesis, sporulation, fruiting body synthesis, cell division, energy metabolism, respiration, cytoskeletal structure synthesis and maintenance, nucleotide metabolism, nitrogen metabolism, carbon metabolism and apoptosis.

23. The method of claim 20, wherein said pathogen is selected from the group consisting of biotrophic, necrotrophic, and hemibiotrophic fungi.

24. The method of claim 20, wherein the polynucleotide functions upon being taken up by the pathogen to suppress a gene that performs a function essential for pathogen survival or growth, said function being selected from the group consisting of ion regulation and transport, enzyme synthesis, nutrient assimilation, viability of the pathogen, sexual reproduction by the pathogen, maintenance of cell membrane potential, amino acid biosynthesis, amino acid degradation, development and differentiation, infection, penetration, development of appressoria or haustoria, mycelial growth, fruiting body growth; sporulation; melanin synthesis, toxin synthesis, siderophore synthesis, sporulation, fruiting body synthesis, cell division, energy metabolism, respiration, cytoskeletal structure synthesis and maintenance, nucleotide metabolism, nitrogen metabolism, carbon metabolism and apoptosis.

25. A method for improving the yield of a crop produced from a crop plant subjected to fungal or oomycete infection, said method comprising the steps of
a) introducing a polynucleotide according to claim 1 into said crop plant,
b) cultivating the crop plant to allow the expression of said polynucleotide, wherein expression of the polynucleotide inhibits fungal or oomycete infection or growth and loss of yield due to fungal or oomycete infection.

26. The method of claim 25, wherein expression of the polynucleotide produces an RNA molecule that suppresses at least a first target gene in a fungal or oomycete plant pathogen that has contacted a portion of said crop plant, wherein the target gene performs at least one essential function selected from the group consisting of ion regulation and transport, enzyme synthesis, nutrient assimilation, viability of the pathogen, sexual reproduction by the pathogen, maintenance of cell membrane potential, amino acid biosynthesis, amino acid degradation, development and differentiation, infection, penetration, development of appressoria or haustoria, mycelial growth, fruiting body growth; sporulation; melanin synthesis, toxin synthesis, siderophore synthesis, sporulation, fruiting body synthesis, cell division, energy metabolism, respiration, cytoskeletal structure synthesis and maintenance, nucleotide metabolism, nitrogen metabolism, carbon metabolism and apoptosis.

27. The method of claim 26, wherein the pathogen is an ascomycete, a basidiomycete, a deuteromycete, or an oomycete.

28. The method of claim 27, wherein the pathogen is a rust fungus.

29. The method of claim 28, wherein the rust fungus is Phakopsora pachyrizi.

30. A method of producing a commodity product comprising obtaining a plant according to claim 11 or a part thereof, and preparing a commodity product from the plant or part thereof.

31. A method of producing food or feed, comprising obtaining a plant according to claim 11 or a part thereof and preparing food or feed from said plant or part thereof.

32. The method of claim 31, wherein the food or feed is defined as oil, meal, protein, starch, flour or silage.

33. A method for suppressing expression of a target gene in a fungal or oomycete cell, the method comprising:
(a) transforming a plant cell with a vector comprising a nucleic acid sequence encoding a dsRNA operatively linked to a promoter and a transcription termination sequence;
(b) culturing the transformed plant cell under conditions sufficient to allow for development of a plant cell culture comprising a plurality of transformed plant cells;
(c) selecting for transformed plant cells that have integrated the vector into their genomes;
(d) screening the transformed plant cells for expression of the dsRNA encoded by the vector;
(e) selecting a plant cell that expresses the dsRNA; and
(f) optionally regenerating a plant from the plant cell that expresses the dsRNA; whereby expression of the gene in the plant is sufficient to modulate the expression of a target gene in a fungal or oomycete cell that contacts the transformed plant or plant cell.
